(19) United States
(12) Patent Application Publication
(10) Pub. No.: US 2021/0023316 A1
(43) Pub. Date: Jan. 28, 2021

(54) METHOD AND INHALER FOR PROVIDING TWO OR MORE SUBSTANCES BY INHALATION

(71) Applicant: Syqe Medical Ltd., Tel-Aviv (IL)

(72) Inventors: Aaron SCHORR, Lotem (IL); Tal LUPO, Yavne (IL); Asaf KROLL, Hofit (IL); Binyamin SCHWARTZ, Sde Eliezer (IL); Nimrod RESHEF, LeHavim (IL); Daniella ATZMONY, Modiin-Maccabim-Reut (IL)

(73) Assignee: Syqe Medical Ltd., Tel-Aviv (IL)

(21) Appl. No.: 16/969,612

(22) PCT Filed: Feb. 14, 2019
(86) PCT No.: PCT/IL2019/050178

§ 371 Date: Aug. 13, 2020

Related U.S. Application Data
(60) Provisional application No. 62/802,737, filed on Feb. 8, 2019.
(60) Provisional application No. 62/630,850, filed on Feb. 15, 2018.

(51) Int. Cl. A61M 15/00; G16H 10/20; G16H 70/40; G16H 20/90; G16H 20/17; G16H 50/70; G16H 20/13; G16H 40/67; A61M 15/06; G16H 40/40; A61K 31/352; A61K 31/05; A24F 40/65; A24F 40/60; A24F 40/50

(52) U.S. Cl. CPC A61M 15/0003; A61M 2205/3368; G16H 70/40; G16H 20/90; G16H 20/17; A61M 15/002; G16H 50/70; G16H 20/13; G16H 40/67; A61M 15/06; G16H 40/40; A61M 15/0021; A61K 31/352; A61K 31/05; A24F 40/65; A61M 2205/505; A61M 2230/04; A61M 2205/3553; A61M 2205/3569; A61M 2205/3331; A61M 2205/3653; A24F 40/60; A24F 40/50; G16H 10/20

(57) ABSTRACT

Some embodiments relate to a method for providing, during a single inhalation from an inhaler, a controllable ratio between at least a first active substance and a second active substance provided from the same source material, comprising: defining a ratio between the first and second active substances to be delivered to a user; setting operation parameters in accordance with the ratio, the operation parameters including at least one of a heating profile of the source material and an airflow profile through the source material; and operating the inhaler according to the operation parameters to deliver to the user, during a single inhalation, at least the first and second active substances at the defined ratio.

### RELATED APPLICATION/S

This application claims the benefit of priority under 35 USC § 119(e) of U.S. Provisional Patent Application No. 62/630,850 filed Feb. 15, 2018 and U.S. Provisional Patent Application No. 62/802,737 filed Feb. 8, 2019, the contents of which are incorporated herein by reference in their entirety.

### FIELD AND BACKGROUND OF THE INVENTION

The present invention, in some embodiments thereof, relates to pulmonary delivery of active substances to a user and, more particularly, but not exclusively, to controlled delivery of two or more active substances provided from the same source material.

Publication WO 2005/072719 A1 to Korthout et al. discloses “The invention relates to an acidic cannabinoid for medical use and to a cannabis extract comprising an acidic cannabinoid. The extract may comprise one or more compounds selected from the group consisting of cannabidiolic acid (CBD-A), cannabidiol (CBD), cannabigerolic acid (CBGA), cannabigerol (CBG), cannabinolic acid (CBN-A) and cannabinol. The invention further relates to a method for preparing a preparation comprising extracting an acidic cannabinoid from cannabis.” (Abstract).

The publication “Can You Pass the Acid Test? Critical Review and Novel therapeutic Perspectives of D9-Tetrahydrocannabinolic Acid A”, Moreno-Sanz, Cannabis and Cannabinoid Research Volume 1.1, 2016 DOI: 10.1089/can.2016.0008 discloses: “D9-tetrahydrocannabinolic acid A (THCA-A) is the acidic precursor of D9-tetrahydrocannabinol (THC), the main psychoactive compound found in Cannabis sativa. THCA-A is biosynthesized and accumulated in glandular trichomes present on flowers and leaves, where it serves protective functions and can represent up to 90% of the total THC contained in the plant. THCA-A slowly decarboxylates to form THC during storage and fermentation and can further degrade to cannabinol.

Decarboxylation also occurs rapidly during baking of edibles, smoking, or vaporizing, the most common ways in which the general population consumes Cannabis. Contrary to THC, THCA-A does not elicit psychoactive effects in humans and, perhaps for this reason, its pharmacological value is often neglected. In fact, many studies use the term “THCA” to refer indistinctly to several acid derivatives of THC. Despite this perception, many in vitro studies seem to indicate that THCA-A interacts with a number of molecular targets and displays a robust pharmacological profile that includes potential anti-inflammatory, immunomodulatory, neuroprotective, and antineoplastic properties. Moreover, the few in vivo studies performed with THCA-A indicate that this compound exerts pharmacological actions in rodents, likely by engaging type-1 cannabinoid (CB1) receptors. Although these findings may seem counterintuitive due to the lack of cannabinoid-related psychoactivity, a careful perusal of the available literature yields a plausible explanation to this conundrum and points toward novel therapeutic perspectives for raw, unheated Cannabis preparations in humans.” (Abstract)

### SUMMARY OF THE INVENTION

According to an aspect of some embodiments there is provided a method for providing, during a single inhalation from an inhaler, a controllable ratio between at least a first active substance and a second active substance provided from the same source material, comprising: defining a ratio between the first and second active substances to be delivered to a user; setting operation parameters in accordance with the ratio, the operation parameters including at least one of a heating profile of the source material and an airflow profile through the source material; and operating the inhaler according to the operation parameters to deliver to the user, during a single inhalation, at least the first and second active substances at the defined ratio.

In some embodiments, defining includes defining an amount of at least the first substance to be delivered to the user in the inhalation.

In some embodiments, defining includes defining a ratio between the first and second active substances.

In some embodiments, a change in operation parameters changes an efficiency of at least one of: vaporization of at least one of the first active substance and the second active substance, and a chemical modification of at least one of the first active substance and the second active substance.

In some embodiments, the second substance is a chemical derivative of the first substance.

In some embodiments, the source material includes at least one of: a botanical material (e.g. plant and/or fungus), two or more different botanical materials, a synthetic material, a synthetic material and a botanical material.

In some embodiments, the first substance is at least one of: Δ9-tetrahydrocannabinol (Δ9-THC), tetrahydrocannabinol acid (THCA) and Cannabidiol (CBD) and the second substance is at least one of a cannabinoid different than the first substance and a terpene.

In some embodiments, the first substance is at least one of: tetrahydrocannabinolic acid (THCA) and Δ9-tetrahydrocannabinol (Δ9-THC), cannabidiolic acid (CBDA) and the second substance is at least one of: tetrahydrocannabinol (Δ9-THC) and Cannabidiol (CBD).

In some embodiments, defining is between at least two different ratios, all of which are configured to deliver to the user the same amount of the first substance.

In some embodiments, the source material comprises a plurality of distinct portions and wherein each of the operation profiles is a combination of all operation profiles applied to each of the distinct portions.

In some embodiments, the operation profile applied to each portion is selected to deliver a predetermined ratio between the first and second active substances.

In some embodiments, all portions have the same source material.

In some embodiments, the heating profile is set to release the first active substance and the second active substance simultaneously into aerosol formed by the inhaler.

According to an aspect of some embodiments there is provided an inhaler for delivering at least two active substances released from a source material to an inhaling user, the inhaler comprising: a controller pre-programmed with and/or in communication with a storage including a plurality of operation profiles; and at least one of: at least one conductor configured to supply sufficient energy for heating the source material when the source material is received within the inhaler; and an airflow system for passing airflow through the source material and for delivering at least two active substances released from the source material to an inhaling user; wherein each of the plurality of operation profiles is associated with a different ratio between the at least two active substances.

In some embodiments, each of the operation profiles is associated with a different therapeutic and/or psychoactive and/or adverse effect the ratio is expected to have on the user.

In some embodiments, the different effect includes a different magnitude of a similar effect. In some embodiments, the inhaler comprises a user interface configured to receive feedback input from the user and to automatically select an operation profile in response to the feedback input.

In some embodiments, the controller is configured for setting two or more operation profiles for use during a single inhalation, the two or more operation profiles selected according to amounts of the active substances or a ratio between the active substances to be released from the source material and delivered to the user.

In some embodiments, the two or more operation profiles are configured to deliver the same amount of a first active substance to the user.

In some embodiments, the storage is configured on a cellular phone application.

In some embodiments, the storage is cloud based.

In some embodiments, each of the plurality of operation profiles is configured to operate within a single inhalation.

According to an aspect of some embodiments there is provided a method for delivering to a user, via an inhaler, a known ratio between at least one cannabinoid and the acidic form of that cannabinoid, the method comprising: defining a ratio to be delivered of the at least one cannabinoid and the acidic form of that cannabinoid; setting operation parameters in accordance with the defined ratio, the operation parameters including at least one of a heating-time profile and an airflow-time profile to be applied to a source material containing at least the acidic form of the cannabinoid; operating the inhaler according to the operation parameters to deliver to the user the at least one cannabinoid and the acidic form of that cannabinoid at the defined ratio.

In some embodiments, the at least one cannabinoid is Δ9-THC and the acidic form of that cannabinoid is THCA

In some embodiments, the at least one cannabinoid is CBD and the acidic form of that cannabinoid is CBDA

In some embodiments, the ratio is defined individually per the user.

In some embodiments, the ratio is defined per a treatment plan.

In some embodiments, the ratio is defined according to a desired therapeutic effect.

In some embodiments, the method comprises setting operation parameters which increase vaporization of the acidic form before decarboxylation.

In some embodiments, increasing the rate of airflow through the source material, while maintaining other parameters constant, increases the ratio between the acidic form and the total of: the at least one cannabinoid and the acidic form of that cannabinoid.

In some embodiments, raising the heating temperature, while maintaining other parameters constant, reduces the ratio between the acidic form and the total of: the at least one cannabinoid and the acidic form of that cannabinoid.

In some embodiments, lengthening the heating duration, while maintaining other parameters constant, reduces the ratio between the acidic form and the total of: the at least one cannabinoid and the acidic form of that cannabinoid.

According to an aspect of some embodiments there is provided an inhaler comprising:

a controller pre-programmed and/or in communication with a storage including: (a) a pre-determined amount of a first active substance released from a source material to be delivered to a user; (b) a plurality of operation profiles, each operation profile associated with a different ratio between the first active substance and at least a second different active substance released from the same source material; one or more heating elements configured for heating the source material when the source material is received within the inhaler; an airflow system for passing airflow through the source material and for delivering at least the first and second active substances released from the source material to an inhaling user.

In some embodiments, the pre-determined amount of the first active substance is a constant amount for each of the plurality of operation profiles.

In some embodiments, the pre-determined amount comprises a total amount of the first active substance to be delivered to the user over multiple inhalations, each inhalation including a pre-determined sub amount.

In some embodiments, the source material comprises a plurality of distinct portions and wherein each of the operation profiles is a combination of all operation profiles applied to each of the distinct portions.

In some embodiments, the operation profile applied to each portion is selected to deliver a predetermined ratio between the first and second active substances.

In some embodiments, all portions have the same source material.

According to an aspect of some embodiments there is provided an inhaler comprising:

a controller pre-programmed and/or in communication with a storage including: (a) a pre-determined amount of a first active substance released from a source material; (b) a plurality of operation profiles, each operation profile associated with a different composition of active substances released from the source material, wherein each composition includes the first active substance at the pre-defined amount, and one or more other active substances at amounts that vary in response to selection of an operation profile; and at least one conduit for delivering the released active substances at the selected composition to an inhaling user.

According to an aspect of some embodiments there is provided a method for delivering to a user, via an inhaler, at least one cannabinoid and the acidic form of that cannabinoid released from a source material, the method comprising: defining a decarboxylation efficiency; controlling at least one of: airflow through the source material; and heating of the source material according to a temperature gradient; and delivering the at least one cannabinoid and the acidic form of that cannabinoid at a ratio set by the defined decarboxylation efficiency.

In some embodiments, controlling heating comprises heating at least a portion of a heating element of source material to a first temperature, and controlling heating to reduce the temperature to a second temperature.

In some embodiments, the method comprises terminating heating upon reaching the second temperature.

According to an aspect of some embodiments there is provided a method for selecting operation parameters of an inhaler for delivering to a user at least one cannabinoid and the acidic form of that cannabinoid released from a source material, comprising: defining a decarboxylation efficiency; selecting at least one of: an airflow profile through the source material and a heating profile of the source material; wherein the selecting takes into account a particle size and density of the source material, and the defined decarboxylation efficiency.

According to an aspect of some embodiments, there is provided a method of providing two or more substances, comprising: providing a defined source material to an inhaler; and applying a selected operation profile to the defined source material, the operation profile designed to deliver from the defined source material a first substance and a second substance at a predetermined ratio associated with the selected operation profile, wherein, the operation profile comprises at least one of: a temperature-time profile and an airflow-time profile, wherein changing the operation profile affects the ratio.

In some embodiments, the operation profile is designed to deliver from the defined source material a selected predetermined amount of the first substance and/or the second substance.

In some embodiments, the method comprises selecting the operation profile from a plurality of operation profiles, wherein each of the operation profiles in the plurality is associated with a different ratio between the first and the second substances.

In some embodiments, the method comprises selecting a subset of operation profiles from the plurality of operation profiles, wherein each one of the operation profiles in the subset of operation profiles is configured to deliver the same predetermined amount of the first substance.

In some embodiments, the operation profile is selected to controllably cause a production of the first substance from at least a portion of the second substance.

In some embodiments, the method comprises adjusting the predetermined ratio by adjusting at least one of: the temperature-time profile and the airflow-time profile.

In some embodiments, the defined source material includes at least one of: a botanical material (e.g. plant and/or fungus), two or more different botanical materials, a mixture of two or more different botanical materials, a synthetic material, a synthetic material and a botanical material and a mixture of a synthetic material and a botanical material.

In some embodiments, the first substance is at least one of: Δ9-tetrahydrocannabinol (Δ9-THC) and Cannabidiol (CBD) and the second substance is at least one of: Tetrahydrocannabinolic acid (THCA) and Cannabidiolic Acid (CBDA).

In some embodiments, the defined source material is at least one of: a solid material, a liquid material and a mixture of solid and liquid materials.

In some embodiments, the method comprises receiving at least one signal indicative of a condition of a user taking the first and second substances; and selecting the operation profile is based on the signal.

In some embodiments, the signal is a feedback signal indicative of an effect of at least one previous inhalation

In some embodiments, the signal is a user request.

In some embodiments, selecting operation profile is according to external data.

In some embodiments, the signal is received from the user.

In some embodiments, the signal is received from an external device.

In some embodiments, the signal is received automatically.

In some embodiments, the signal is related to a medical condition of the user.

In some embodiments, the signal is related to a general feeling of the user.

According to an aspect of some embodiments there is provided a method of controlling an inhaler, comprising: receiving a selected operation profile associated with the delivery of a first substance and a second substance from a defined source material at a predetermined ratio; and activating the inhaler to apply the selected operation profile to the defined source material when located in the inhaler, wherein, the operation profile comprises at least one of: a temperature-time profile and an airflow-time profile.

In some embodiments, the method comprises receiving at least one signal indicative of a condition of a user taking the first and second substances; and selecting the operation profile is based on the signal.

According to an aspect of some embodiments there is provided a server computer comprising: a database storing thereon data indicative of a plurality of operation profiles, wherein each of the operation profiles is designed to deliver from a source material a first substance and a second substance at a predetermined ratio, and each operation profile comprises at least one of: a temperature-time profile and an airflow-time profile; and a processor configured to: receive from a first remote device data indicative of a predetermined ratio between the first and the second substances; select an operation profile from the stored plurality of operation profiles that is designed to provide the predetermined ratio; and send the selected profile to a second (possibly the same as the first) remote device which is configured to enable the inhaler to apply the selected operation profile to the defined source material.

In some embodiments, the remote device is the inhaler.

In some embodiments, the remote device is a mobile device associated with the inhaler.

According to an aspect of some embodiments there is provided an inhaler for providing two or more vaporized substances, comprising: a heating operator for heating one or more vaporization chambers in the inhaler; an airflow system for providing airflow to the one or more vaporization chambers; and a controller configured to: control at least one of: the heating operator and the airflow system to apply an operation profile to a source material when located in an vaporization chamber in the inhaler; wherein, the operation profile comprises at least one of: a temperature-time profile and an airflow-time profile, and wherein the operation profile is designed to deliver from a defined source material a first substance and a second substance at a predetermined ratio.

In some embodiments, the heating activator comprises a power source for providing electrical power to a heating element.

In some embodiments, the power source is configured to provide the electrical power to a heating element being in contact with the source material.

In some embodiments, the heating operator further comprises the heating element.

In some embodiments, the heating operator comprises a heating element for heating the provided airflow.

In some embodiments, the inhaler comprises a user interface, and the controller is configured to: display, on a display of the user interface, two or more indicators each associated with a different operation profile; receive, from the user interface a section of the first operation profile.

According to an aspect of some embodiments there is provided a method of providing two or more substances, comprising: providing a defined source material to an inhaler; accessing data indicative of a plurality of operation profiles, each configured to deliver a predetermined amount of a first substance and to deliver at least one second substance from the defined source material, wherein the plurality of operation profiles vary in the amount the second substance delivered; applying a selected first operation profile from the plurality of operation profiles to the source material.

In some embodiments, the plurality of operation profiles vary in at least one of: a temperature-time profile and an airflow-time profile.

In some embodiments, the plurality of operation profiles vary from one another in the effect that giving the two or more source materials by applying the profile has on a user, and the method comprises: selecting the first operation profile based on the effect.

In some embodiments, the defined source material has a defined content at a defined weight.

In some embodiments, the defined source material includes at least one of: a botanical material, two or more different botanical materials (e.g. having the same plant variety but a different chemical finger print), a mixture of two or more different botanical materials, a synthetic material, a synthetic material and a botanical material and a mixture of a synthetic material and a botanical material.

In some embodiments, the defined source material comprises Cannabis and the first substance is selected from a group consisting of: Δ9-tetrahydrocannabinol (Δ9-THC), Cannabidiol (CBD), Tetrahydrocannabinolic acid (THCA) and Cannabidiolic Acid (CBDA).

In some embodiments, the second substance is at least one of: Tetrahydrocannabinolic acid (THCA) and Cannabidiolic Acid (CBDA), Cannabinol (CBN), Cannabigerol (CBG), Cannabichromene (CBC), Cannabicyclol (CBL), Cannabivarin (CBV), Tetrahydrocannabivarin (THCV), Cannabichromevarin (CBCV), Cannabigerovarin (CBGV), Cannabigerol Monomethyl Ethe (CBGM), Cannabielsoin (CBE) and Cannabicitran (CBT).

In some embodiments, the method comprises receiving a selection of the first operation profile.

In some embodiments, receiving the selection is from at least one of: a user interface of the inhaler, a mobile device associated with the inhaler and a remote processor communicating with the inhaler.

In some embodiments, the method comprises receiving a first signal indicative of a condition of a user.

In some embodiments, the method comprises selecting the first operation profile based on the received first signal.

In some embodiments, receiving the first signal is following the application of the first operation profile.

In some embodiments, the method further comprises selecting a second activation profile from the plurality of activation profiles based on the received first signal. In some embodiments, the first signal is received from the user.

In some embodiments, the first signal is received from an external device associated with the user.

In some embodiments, the signal is received automatically.

In some embodiments, the first signal is related to a medical condition of the user.

In some embodiments, the signal is related to a general feeling of the user.

In some embodiments, the method comprises providing the second operation profile to the source material, receiving a second signal following the application of the second operation profile; and selecting an operation profile from the plurality of operation profiles based on the received first and second signals.

In some embodiments, at least some of the operation profiles in the plurality of operation profiles are configured to deliver a first predetermined amount of a first substance, a second predetermined amount of a second substance and at least one third substance from the source material.

According to an aspect of some embodiments there is provided a method of controlling an inhaler, comprising: receiving a selected operation profile from a plurality of operation profiles, each configured to deliver a predetermined amount of a first substance and to deliver a second substance from a defined source material, wherein the plurality of operation profiles vary in the amount the second substance delivered; and activating the inhaler to apply the selected operation profile to the defined source material when located in the inhaler, wherein, the operation profile comprises at least one of: a temperature-time profile and an airflow-time profile.

In some embodiments, receiving the selected operation profile is from at least one of: a user interface of the inhaler, a mobile device associated with the inhaler and a remote processor communicating with the inhaler.

According to an aspect of some embodiments there is provided an inhaler for providing two or more vaporized substances, comprising: a heating operator for providing heat to one or more vaporization chambers in the inhaler; an airflow system for providing airflow to the one or more vaporization chambers; and a controller configured to: access data indicative of a plurality of operation profiles, each configured to deliver a predetermined amount of a defined substance and at least one other substance from a source material, control at least one of: the heating operator and the airflow system to provide a selected first operation profile from the plurality of operation profiles to a source material located at an vaporization chamber, wherein, the operation profile comprises at least one of: a temperature-time profile and an airflow-time profile.

In some embodiments, the heating operator comprises a power source for providing electrical power to a heating element.

In some embodiments, the heating operator further comprises the heating element.

In some embodiments, the power source is configured to provide the electrical power to a heating element being in contact with the source material.

In some embodiments, the heating activator comprises a heating element for heating the provided airflow.

In some embodiments, the operation profiles in the plurality of operation profiles vary from one another in at least one of: the temperature-time profile and the airflow-time profile.

In some embodiments, the inhaler comprises a user interface, and the controller is configured to: display, on a display of the user interface, two or more indicators each associated with a different operation profile; receive, from the user interface a section of the first operation profile.

According to an aspect of some embodiments there is provided a method of providing two substances by inhalation, comprising providing a source material comprising a first substance that undergoes modification to form a second substance when exposed to heat; applying an operation profile to cause vaporization of the first substance and a delivery of the first substance to a user; wherein the operation profile further causes a delivery of a predetermined amount of the second substance together with the first substance, wherein, the operation profile comprises at least one of: a temperature-time profile and an airflow-time profile.

In some embodiments, the operation profile causes the delivery of the first substance at a predetermined amount.

In some embodiments, the source material comprises of a mixture of two or more substances, one of them being the first substance.

In some embodiments, the source material includes at least one of: a botanical material, two or more different botanical materials, a mixture of two or more different botanical materials, a synthetic material, a synthetic material and a botanical material and a mixture of a synthetic material, and a botanical material.

In some embodiments, the source material is in a liquid phase.

In some embodiments, the source material is a mixture of solid and liquid.

In some embodiments, the first substance is Tetrahydrocannabinolic acid (THCA) and the second substance is Δ9-tetrahydrocannabinol (Δ9-THC).

In some embodiments, the first substance is Cannabidiolic Acid (CBDA) and the second substance is Cannabidiol (CBD).

According to an aspect of some embodiments there is provided an inhaler for providing two or more vaporized substances, comprising: a heating operator for heating one or more vaporization chambers in the inhaler; an airflow system for providing airflow to the one or more vaporization chambers; and a controller configured to: control at least one of: the heating operator and the airflow system to apply an operation profile to a source material when located in a vaporization chamber in the inhaler; wherein, the operation profile comprises at least one of: a temperature-time profile and an airflow-time profile, and wherein the operation profile is designed to deliver from a defined source material a first substance and a predetermined amount of a second substance, the first substance undergoes modification to form the second substance when exposed to heat.

In some embodiments, the heating activator comprises a power source for providing electrical power to a heating element.

In some embodiments, the power source is configured to provide the electrical power to a heating element being in contact with the source material.

In some embodiments, the heating operator further comprises the heating element.

In some embodiments, the heating activator comprises a heating element for heating the provided airflow.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

Some embodiments of the invention, both as to organization and method of operation, together with objects, features, and advantages thereof, may best be understood by reference to the following detailed description when read with the accompanying drawings in which:

FIG. 1A is a high-level block diagram of an inhaler according to some embodiments of the invention;

FIG. 1B is an illustration of an inhaler according to some embodiments of the invention;

FIGS. 1C and 1D are a side view and a top view of a chip for holding a source material according to some embodiments of the invention;

FIG. 2 is a flowchart of a method of providing substances to a user according to some embodiments of the invention;

FIG. 3 is a flowchart of a method of providing substances to a user according to some embodiments of the invention;

FIG. 4 is a flowchart of a method of providing substances to a user by inhalation according to some embodiments of the invention;

FIG. 5 is a bar chart presenting ratios between total THC/CBD received at various operation profiles according to some embodiments of the invention;

FIG. 6 is a bar chart presenting ratios between THCA/Δ9-THC and CBDA/CBD at various operation profiles according to some embodiments of the invention;

FIG. 7 is a graph showing influence of temperature applied by different operation profiles on decarboxylation of THCA to Δ9-THC according to some embodiments of the invention;

FIGS. 8A-8D are graphical representations of heating-time profiles according to some embodiments of the invention;

FIGS. 9A-9D are graphical representations of temperature-time profiles according to some embodiments of the invention;

FIG. 10 is a flowchart of a method for delivering selected amounts and/or a selected ratio of THCA and Δ9-THC to an inhaling user, according to some embodiments;

FIG. 11 schematically illustrates an inhaler for providing one or more substances released from source material to a user, according to some embodiments;

FIG. 12 is a diagram of parameters and conditions which affect decarboxylation of cannabinoids, according to some embodiments;

FIG. 13 is an example of a timeline for delivering using a selected operation profile one or more substances to a user, according to some embodiments;

FIG. 14 is a flowchart of an operation profile in which heating is controlled according to a temperature gradient, according to some embodiments;

FIG. 15 is a schematic diagram of an inhaler configured to deliver, optionally in a single inhalation of the user, two or more active substances at predefined amounts and/or at a known ratio, according to some embodiments;

FIG. 16 schematically illustrates source material chips comprising individually controllable portions, according to some embodiments; and

FIGS. 17A-B are graphs presenting experimental results, performed in accordance with some embodiments.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated in several figures to indicate corresponding or analogous elements.

### DESCRIPTION OF SPECIFIC EMBODIMENTS OF THE INVENTION

The present invention, in some embodiments thereof, relates to pulmonary delivery of active substances to a user and, more particularly, but not exclusively, to controlled delivery of two or more active substances provided, optionally during a single inhalation, from a source material.

An aspect of some embodiments relates to controlled vaporization and delivery of selected amounts of two or more active substances from a source material, optionally delivered to an inhaling user simultaneously, for example, during a single inhalation.

In some embodiments, one of the active substances is a chemical derivative of the other substance. In some embodiments, one of the active substances is released in its natural form, and another active substance is released in a chemically modified form.

In some embodiments, amounts of two or more of the substances and/or a ratio between substances are selected prior to delivery. In some embodiments, an operation profile which defines parameters such as a duration of heating the source material, a heating temperature, a rate of airflow through the source material and/or other operational parameter is selected in accordance with the predetermined amounts or ratio to be delivered. Optionally, the source material comprises a plurality of spatially defined individually controlled sections and the operation profile includes a spatial definition of the operation profile, which define at least one of which section to use, when and which operation profile to apply to each section.

In some embodiments, the amounts and/or ratio are defined directly (e.g. by the user, physician, and/or other related personnel). Additionally, or alternatively, the amounts and/or ratios are not selected directly, and are optionally automatically implemented in response to selection of a desired effect, selection of a treatment regimen, a user preference and/or other input. Optionally, the user does not need to care about or know the specific ratios, except that the ratios of the different options are different and may have a different effect on the user. In some embodiments, the amounts and/or ratio are selected by the system according to, for example, data configured on a device memory, data configured on a remote server, a clinical database, a population database, and/or other source of data.

In some embodiments, a single activation of an inhaler, for example as described herein, generates release of two or more active substances which can then be delivered to the user together in a single inhalation. The active substances may be released simultaneously, in overlapping periods and/or separately, but close enough in timings and short enough in total duration so as to be delivered together. A potential advantage of selecting a ratio between the active substances provided may include inducing, controlling and/or preventing one or more predetermined effects (e.g. a therapeutic effect, a psychoactive effect, a cognitive effect, an adverse effect) on the user.

In some embodiments, operational parameters (e.g. a heating temperature, a heating duration, a rate of airflow through the source material and/or a spatial definition of the source material) are selected to cause an efficiency of one or both of vaporization of an active substance and a chemical modification of an active substance. While in some cases vaporization and chemical modification may occur simultaneously or at least partially overlapping, precise adjustment of operational parameters may affect the ratios between different active substances in the formed aerosol.

In some embodiments, vaporization of a substance and/or a chemical modification of a substance by the device and system described herein may be reached at temperatures different than ones known in literature and/or dictated by nature. In some embodiments, physical system parameters and/or constraints lead to a change in the vaporization and/or chemical modification states of various active substances.

In some embodiments, one or more of the following ratios are controlled: a ratio between at least two substances exiting the source material chip; a ratio between the at least two substances in the formed aerosol; a ratio between the at least two substances when entering the user's mouth; a ratio between the at least two substances when entering the user's respiratory system; a ratio between the at least two substances as expressed by blood concentrations of the user.

In some embodiments, a calibration table and/or physical models are applied to correlate between the ratios at the various stages of delivery. For example, the ratio between the two substances as measured in the aerosol is equivalent to a certain ratio between the same two substances as found in the user's blood, following delivery; the ratio between the two substances as the substances exit the chip is equivalent to a certain ratio of these substances when entering the user's mouth; etc. In an example, a ratio is defined and controlled for two substances immediately upon vaporization of the substances from plant material according to an estimated ratio of these substances in the respiratory system and/or blood circulation of the user. Various methods may be employed for detecting the ratio at each of the above mentioned stages, optionally for correlating between the ratios of different stages. For example, chromatography may be applied to detect the ratio in the aerosol; a blood sample may be tested for detecting the ratio in the blood; a tissue biopsy may be performed for detecting the ratio in tissue.

An aspect of some embodiments relates to pulmonary delivering of at least one neutral cannabinoid and the acidic form of that cannabinoid to an inhaling user. In some embodiments, a decision is made to treat a user with Δ9-THC and/or with its acidic precursor, THCA. In some embodiments, the decision involves setting one or more specific amounts to be delivered. In some embodiments, a delivery schedule is defined, including for example selected amounts to be delivered at selected times. In some embodiments, a THCA/Δ9-THC ratio is set. In some embodiments, an amount and/or type of source material (e.g. a certain cannabis strain or a certain batch of cannabis having a defined chemical composition) are selected according to the ratio to be provided. A potential advantage of the ability to release and deliver a selected ratio of THCA/Δ9-THC to a user may include affecting the entourage, and potentially modulating side effects (e.g. psychoactive effects) which are often associated with delivery of Δ9-THC. In some embodiments, THCA is delivered at an amount sufficient to obtain a predetermined effect on the user, for example, provide pain relief, treat nausea. Optionally, amounts of THCA are to be delivered are calibrated per the specific user, for example based on one or more of: previous usage and effect, feedback obtained from the user, data collected from the user (e.g. via one or more sensors and/or via a user interface and/or via the inhaler itself), optionally in combination with data obtained from other users. In some embodiments, feedback and/or data are collected from the user before, during, and/or after an inhalation session (during which the user inhales a plurality of inhalations from the inhaler). Optionally, feedback and/or data are collected from the user before, during and/or after a single inhalation.

An aspect of some embodiments relates to affecting an entourage of a delivered active substance by selection of device operation profiles. In some embodiments, the device is set to deliver a first active substance (e.g. Δ9-THC or THC, being a total amount of Δ9-THC and THCA) at a predefined amount, while one or more other active substances are delivered in amounts that vary in accordance with the change in operation profile. Optionally, the predefined amount of the first active substance is set to be constant over multiple deliveries, e.g. for multiple inhalations.

In an example, selection of a first operation profile actuates delivery of Δ9-THC or THC at a predefined amount, and one or more other substances (such as THCA, CBD terpenes, etc.) each at a pre-set, controllable ratio with respect to the Δ9-THC (THC); selection of a second operation profile will actuate delivery of Δ9-THC (or THC) at the same predefined amount, and at least one of the other substances (such as THCA, CBD, terpenes, etc.) at a different ratio with the first substance than the ratio of the first operation profile. In some embodiments, the ratio between THC (or Δ9-THC) and at least one of the other substances is known for each of a plurality of operation profiles. Optionally, the difference in ratios or entourage is inferred from the different effects of the aerosols generated by each profile. In some embodiments, different operation profiles induce different effects on the user. In some cases, a different effect may include a similar effect, but having a different magnitude. a variation in the magnitude of psychoactive effect and/or potency of odor and/or degree of symptom relief, etc. Optionally, more than an effect is induced in the user in some operation profile but not in others. For example, in a first operation profile only pain relief is induced with no side effects, but in a second profile a greater degree of pain relief is achieved but accompanied with slight drowsiness and in a third profile pain relief is even greater than in the second, but drowsiness is lesser than in the second operation profile.

In some embodiments, a change in the operation profile may result in a change in the induced effect on the user. For a similar composition of active substances delivered, the effect may vary between individual users; therefore in some embodiments, personal preferences and/or a current condition are taken into account in selecting an operation profile.

In some cases, delivering a first substance in a pre-defined amount, in each operation profile along with at least a second substance which varies in amount, for example varies for different inhalations and/or for different use sessions, leads to a different entourage effect on the user. By modulating the amounts of at least a second substance and/or by modulating a ratio between the first substance which is provided at a predefined amount and at least the second substance, different blood concentrations of these substances may be reached, generating different physiological and/or cognitive effects on the user.

In some embodiments, a device or system for example as described herein recommends a profile, for example by providing the user with information regarding the expected effect (e.g. therapeutic effect, psychotropic effect, adverse effect), a scent, and/or a magnitude of one or more of the aforesaid, and/or other information which may assist a user in determining if the profile and its associated effects suit the user's current need, preference, and/or and condition; additionally or alternatively, the user tests one or more different profiles and determines the effect on herself and/or her personal preferences, according to which future profiles may be automatically suggested to the user. In some embodiments, the controller is programmed with a plurality of different operation profiles, and is configured to implement one or more of the profiles for example in response to defining of a ratio or amounts of substances to be delivered.

In some embodiments, two or more operations profiles are applied to provide the same or different amount of a selected active substance. In some embodiments, operation profiles are applied concomitantly. In some embodiments, an operation profile applied during a single inhalation is comprised of two or more stages, each stage setting different heating parameters and/or different airflow parameters. In some embodiments, operation profiles are applied simultaneously, for example, using an inhaler, which is configured to independently address and release substances from physically separated source materials.

Additionally, or alternatively, in some embodiments, a constant operation profile is used, and a composition of substances released may be varied, for example, by use of source material chips in which the source material includes pre-defined, optionally measured proportions of active substances to be released.

An aspect of some embodiments relates to selection and/or manipulation of physical parameters for controlling a chemical modification or reaction of one or more active substances released from a source material. In some embodiments, parameters are selected and/or modified to release predetermined amounts or ratios of active substances from a source material. In some embodiments, parameters are selected so at to obtain a selected decarboxylation efficiency or range thereof.

In some embodiments, selection of parameters takes into account a vaporization temperature of an active substance. In some embodiments, selection of parameters takes into account a stage (e.g. one or more of a timing, a temperature, a chemical setting) in which a substance goes through a chemical modification or reaction. In an example, vaporization of active substances from cannabis is controlled to reach a state in which THCA is vaporized, yet at least a portion of the THCA does not go through decarboxylation (before, during and/or after vaporization) into Δ9-THC, and can be delivered to the user in the form of THCA.

In some embodiments, selection of parameters comprises selection of conditions which have a thermal effect on active substance release. In some embodiments, selection of parameters comprises selection of conditions, which affect the flow of air through the source material. In some embodiments, parameter sets are selected as a combination of parameters related to a structure and pre-arrangement of the source material, and operational parameters applied during use. In some embodiments, one or more parameters are selected to compensate for others, for example, reducing the heating temperature may be compensated for by a lengthening a duration of heating to reach similar results; an increased density of source material may be compensated for by increasing a rate of airflow, and as such.

In some embodiments, physical parameters related to preparation and pre-arrangement of the source material are selected, including, for example, one or more of a size (e.g. diameter) of the source material particles; a density of the source material; humidity conditions; added chemical substances (e.g. substances which delay or instead accelerate a chemical modification, substances which delay or accelerate vaporization); a carrier material onto which the source material is mounted or embedded.

Additionally or alternatively, physical parameters related to operation are selected, including, for example: a heating temperature; heating duration; cooling duration; a temperature gradient across the source material (e.g. across a thickness of the source material layer); a rate of airflow through the source material; a duration of passing airflow through the source material and/or a spatial definition of the operation profile across a plurality of individually controlled source material portions.

In some embodiments, parameters or parameter sets are selected or modified according to a computational model, a formula, a look up table. Optionally, a known, pre-tested parameter set is selected, for example a parameter set which when applied generates extraction of certain amounts of active substances from a specific source material (e.g. a certain cannabis strain).

In some embodiments, parameter selection takes into account a tradeoff between effects. For example, raising the heating temperature may result in reducing the THCA/Δ9-THC ratio. In some embodiments such raising is selected according to a degree of further oxidization of Δ9-THC. This may be especially noticeable if the source material (e.g. plant material) contains a significant amount of Δ9-THC, prior to applying of heat.

In some embodiments, a condition of the source material, including initial concentrations of active substances, is taken into account when selecting operational parameters. Optionally, the controller receives or reads data pertaining to the initial concentrations of substances in the source material of an inserted chip (e.g. via an electronic tag of the chip, such as RFID), and automatically selects a set of suitable parameters.

In some embodiments, for example when ventilation is forced and airflow is actively passed through the source material, increasing the rate of airflow through the source material may increase the amount of THCA released, but on the other hand an airflow rate increased beyond a certain threshold may not be suitable for inhalation by a frail user. Additionally, or alternatively, flow through the source material is actuated by the user breathing in through the device, and the rate of airflow may be controlled and modified, for example, by use of one or more bypass routes, for example as described hereinbelow.

In some embodiments, a duration of heating and/or a duration of passing airflow are adjusted, for example, reducing the temperature might be compensated for by lengthening the heating duration. In some embodiments, adjustment of a heating duration and/or a duration of passing airflow is performed taking into account that the time period is limited by the length of inhalation of the user, yet some extension of time is permitted within such limit.

An aspect of some embodiments relates to controlled heating of a source material for modifying and/or activating and/or deactivating substances released from the source material. In some embodiments, heat is applied to generate a structural molecular change in the substance, for example, decarboxylation (such as of a cannabinoid acid form to a neutral cannabinoid). In some embodiments, due to that different cannabinoids may have different vaporization temperatures and different decarboxylation temperatures, different decarboxylation efficiencies can be elicited for different cannabinoids. In an example, parameters are selected to generate decarboxylation of only 10%, 5%, 15% or intermediate, higher or lower percentage of THCA found in the source material yet generate decarboxylation of more than 20%, 30%, 40% or intermediate, higher or lower percentage of CBDA found in the same source material.

In some embodiments, a heating profile is controlled to obtain selected decarboxylation efficiency. In some embodiments, heating is to a constant temperature; additionally or alternatively, heating is controlled to generate a temperature gradient. In an example, heating is controlled to heat a heating element in contact with at least a portion of the source material to first temperature, and then to reduce the temperature to second temperature. Optionally, heating is terminated upon reaching the second temperature. A potential advantage of controlling heating according to temperature gradient may include bringing the source material controllably to a state in which decarboxylation occurs substantially at the pre-selected efficiency and maintaining it in that state. In some embodiments, for different temperature gradients, different decarboxylation efficiencies can be reached.

As referred to herein, a “single inhalation” may include a time period of between 0.5-5 seconds, 1-3 seconds, 2-3 seconds or intermediate, longer or shorter time periods during which the user's mouth is placed on the inhaler mouthpiece and the user inhales, whereby at least along a portion of that time the user draws in the vapor/aerosol of the released substances. A “single inhalation” may include a time period through which the user does not exhale.

A potential advantage of delivering two or more active substances in a single inhalation may include ensuring that the user received those two substances simultaneously. In an example, one of the substances has a psychoactive effect, and the other substance contradicts that effect.

In some embodiments a potential advantage of delivering two or more active substances in a single inhalation may include benefiting from an entourage effect of a plurality of substances being provided from the same source material (e.g. from a botanical). Controlling the ratios between such substances allows providing a single source material for inhalation, while enabling a plurality of distinct ratios in the aerosol.

In some embodiments, a defined ratio is reached after a full inhalation duration, for example, during a first portion of the inhalation, a first ratio is reached, and during the second portion of the inhalation, a second ratio is reached, and the two ratios balance to obtain a total ratio that was initially set.

As referred to herein with respect to the experiment results presented, a percentage of THCA that did not decarboxylate was calculated using mg measurements of THCA and Δ9-THC and taking into account a molecular weight, using the following calculation: (THCA/(Δ9-THC+ THCA*0.877) Likewise, s referred to herein with respect to the experiment results presented, a percentage of CBDA that did not decarboxylate was calculated using mg measurements of CBDA and CBD and taking into account a molecular weight, using the following calculation: (CBDA/(CBD+ CBDA*0.878).

As referred to herein, the terms “substance provided” and “substance released” may refer to any substance, formed or modified (e.g. chemically modified, thermally modified) which originates from one or more source materials, optionally to be delivered to the user. In some embodiments, the terms “substance provided” and “substance released” cover a substance that exits the source material chip in one form, and concomitantly or only later changes to a different form.

In some embodiments, a “substance provided” or “substance released” covers a substance that separates from the source material, e.g. by being vaporized from the source material. In some embodiments, the “provided substance” or “released substance” is one that becomes a part of a formed aerosol, optionally to be delivered to the inhaling user.

A “conductor” as referred to herein may include an element configured for generating and/or transferring of energy (such as electrical and/or thermal energy). In some embodiments, the conductor is configured to generate and/or transfer energy at amount sufficient for heating the source material so as to vaporize one or more active substances from the source material. In some embodiments, the conductor conducts electrical current, for example, an electrode and/or an electric wire. In some embodiments, the conductor conducts heat. The term “conductor” is meant to cover means suitable for transferring and/or generating electrical and/or thermal energy.

The benefits of medical cannabis are nowadays well known and medically proven. Active substances such as tetrahydrocannabinol (THC) and cannabidiol (CBD) are known to have therapeutic effects. Cannabis like other medicinal and/or recreational plants should be inhaled to gain the maximal effect from the active materials. Other plants, for example tobacco, are also consumed by inhalation as well as synthetic materials, such as, salbutamol.

Cannabis vapor or aerosol typically contains cannabinoids, terpenes flavonoids and alkaloids as well as other components, while tobacco vapor typically contains alkaloids (for example nicotine) and flavonoids as well as other components. Cannabinoids occur in raw cannabis plant predominantly as acids. For example, tetrahydrocannabinol (THC) is present in the plant mostly as tetrahydrocannabinol acid (THCA) and cannabidiol (CBD) is present in the plant mostly as cannabidiol acid (CBDA). Cannabinoid acids slowly decarboxylate as the plant dries to produce the cannabinoids in their neutral form (e.g. THC forms Δ9-THC). This process is accelerated when the acids are heated (e.g. when delivered by smoking or inhalation or when baked into foodstuffs).

Medical benefits of THC and CBD are well known however, recent researches showed medical benefits of cannabinoid acids as well. For example, some known medical benefits of THCA may include anti-inflammatory, neuro-protective, anti-emetic, and anti-proliferative effects. In yet another example, some known medical benefits of CBDA may include anti-proliferative, anti-inflammatory and anti-nausea effects. Furthermore, CBDA may include antioxidant, Antibacterial/antimicrobial and painkiller elements. Delivery of THCA, for example, is typically performed by ingesting the raw plant (e.g. in a smoothie) and not through inhalation, since heating (for inhalation) causes its decarboxylation.

Before explaining at least one embodiment of the invention in detail, it is to be understood that the invention is not necessarily limited in its application to the details of construction and the arrangement of the components and/or methods set forth in the following description and/or illustrated in the drawings and/or the examples. The invention is capable of other embodiments or of being practiced or carried out in various ways.

In the following detailed description, numerous specific details are set forth in order to provide a thorough understanding of the disclosure. However, it will be understood by those skilled in the art that the disclosed subject matter may be practiced without these specific details. In other instances, well-known methods, procedures, and components have not been described in detail so as not to obscure the present disclosure.

An inhaler according to some embodiments of the invention may allow to controllably deliver two or more substances during a single inhalation, from a source material. In some embodiments, in order to controllably deliver the two or more substances a predetermined operation profile may be applied by the inhaler. The predetermined operation profile may include at least one of: a temperature-time profile and an airflow-time profile for controlling the amount of heat provided. Such an operation profile may be selected for example to control an amount of energy provided to the source material and/or the amount of energy delivered to the substances. Optionally, the source material comprises a plurality of spatially defined individually controlled sections and an operation profile includes a spatial definition of the operation profile, which define at least one of which section to use, when and what operation profile to apply to each section.

A source material, according to embodiments of the invention, may include the two or more substances. In some embodiments, the source material may include at least one substance in its original form (e.g., nicotine, a cannabinoid, and/or salbutamol) to be vaporized, volatilized, aerosolized, and/or otherwise released from the source material by the inhaler. In some embodiments, the source material may include a material that undergoes modification (e.g., decarboxylation) before, during and/or after vaporization.

A source material, according to embodiments of the invention, may include at least one of: a botanical material (e.g. plant and/or fungus), two or more different botanical materials (e.g., placed in different locations of a holder), a mixture of two or more different botanical materials, a synthetic material, a synthetic material and a botanical material (e.g., placed in different locations of holder), a mixture of a synthetic material and a botanical material and any combination thereof.

The source material may comprise solid, liquid or gel, or of a mixture of solid, liquid and/or gel. For example, the source material may be or may include chopped plant material (e.g., leaves, flowers, bark, stems, grains, fruits and/or fungi). In yet some examples, the source materials may include a synthetic material such as salbutamol or morphine. In yet some examples, the source material may include an extract (e.g. liquid or solid) of a substance from a botanical material (whether or not purified), for example, an aromatic oil, an extracted powder and the like. In some embodiments, the source material may include at least one botanical material. Non-limiting examples of such botanical materials include cannabis flowers and tobacco leaves. A source material according to some embodiments of the invention may include at least one volatile substance, vaporizable substance, evaporable substance, aerosolizable substance and the like.

As used herein a “temperature-time profile” may include the temperature as a function of time of the source material when placed in the inhaler. The temperature-time profile may be controlled by controlling the amount and/or duration of heat or energy provided to the source material and affected and/or effected by airflow provided to the source material. A temperature-time profile may be measured by placing a sensor (e.g. IR sensor, a thermocouple, an impedance sensor and the like) in proximity to the source material and/or by sensing a temperature of heated air before and/or after contacting the source material. Temperature sensing may be used as real time feedback to a controller, for controlling the application of an operation profile to the source material. In some embodiments, the temperature-time profile may include one or more profiles of one or more controllable parameters that may affect the temperature-time profile. For example, the temperature-time profile may include a power-time profile (or a current-time profile) of the power (or current) supplied to heating elements that may heat the source material. In yet another example, the temperature-time profile may include the power-time profile and an airflow-time profile (as disclosed herein) that may both effect the temperature-time profile.

As used herein an “airflow-time profile” may include a change over time of the amount and/or velocity (e.g., the air capacity) and/or pressure of air provided to a source material placed in the inhaler. This air may be in ambient temperature and/or may be temperature controlled (e.g. heated) before contacting the source material. An airflow-time profile may be measured by placing a sensor of a property that is indicative of the profile (e.g. a pressure sensor) in proximity to the source material and/or in other locations along the path of airflow within the inhaler device.

In some embodiments, changing at least one parameter in the predetermined operation profile may change the amount of at least one substance delivered from a source material. In some embodiments, the inhaler may deliver the two substances at a predetermined ratio, for example, deliver both Δ9-THC and THCA at a predetermined ratio (e.g., known predetermined amounts) at the same inhalation. In some embodiments, changing at least one parameter in the predetermined operation profile may change the ratio between the two substances.

In some embodiments, the inhaler may deliver at least one known substance (e.g., Δ9-THC or THC, being a total of Δ9-THC and THCA) at a predetermined amount and one or more other substances in the same inhalation (e.g., THCA and/or CBD). In some embodiments, changing at least one parameter in the predetermined operation profile may cause the delivery of the same amount of total THC (or Δ9-THC), but with different amounts of THCA and/or CBD.

In some embodiments, selecting the operation profile may allow to control decarboxylation and/or other heat activated reactions. For example, a first operation profile may cause decarboxylation of 80% of THCA to Δ9-THC in cannabis and a second operation profile (optionally an operation profile that applies less heat, and/or one that applies airflow at a higher rate) applied to essentially identical source material may cause decarboxylation of only 50% of the THCA.

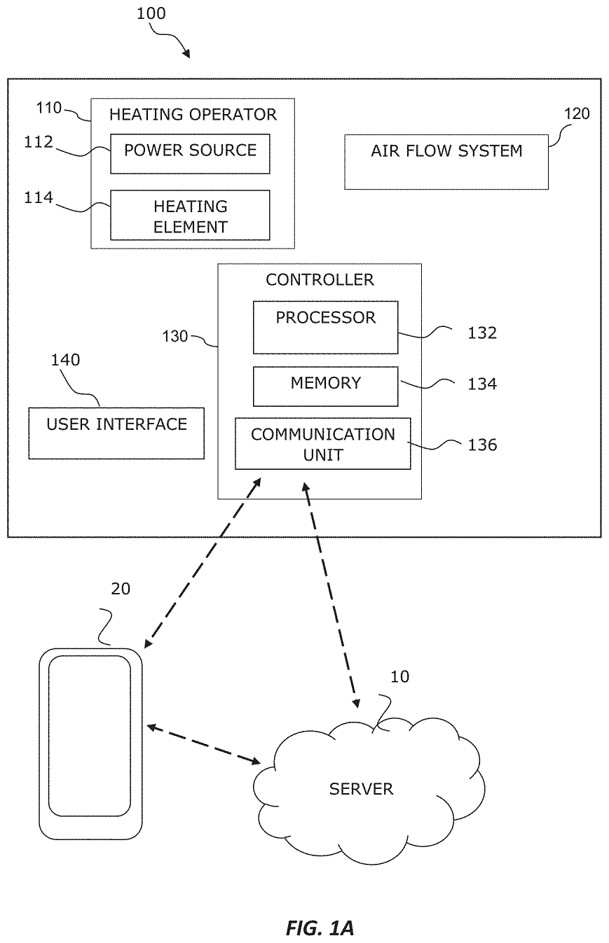

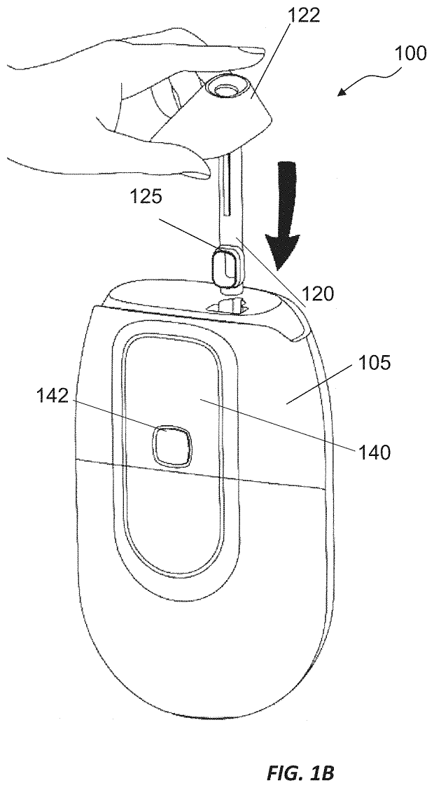

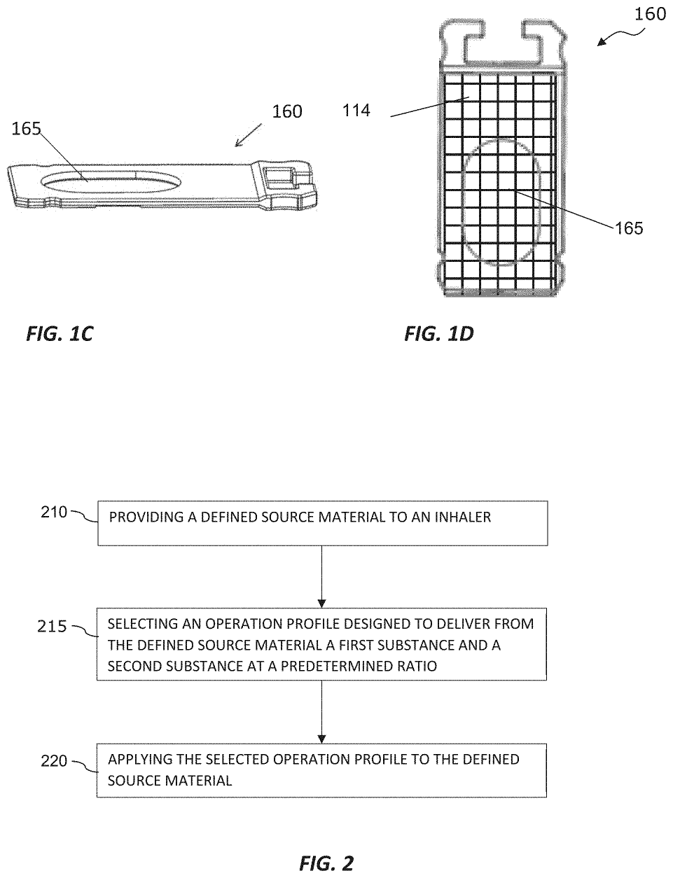

Reference is now made to FIG. 1A which is a high-level block diagram of an inhaler according to some embodiments of the invention. An inhaler 100 may include a heating operator 110, an airflow system 120 and a controller 130. Inhaler 100 may include a body 105 for housing the various components of inhaler 100 (illustrated in FIG. 1B). The housing may include a vapor chamber in which vapors are being produced and a vapor outlet (which may include a nozzle or mouthpiece) for delivering the vapor to a user. Inhaler 100 may include one or more vaporization chambers for placing a source material. In some embodiments, the source material (e.g., chopped botanical material) may be placed directly in the vaporization chamber in inhaler 100. In some embodiments, the source material may be included in one or more holders (e.g., chip 160 illustrated in FIGS. 1C and 1D or a capsule or pod or the like) to be placed in the vaporization chamber. In some embodiments, more than one holder may be placed in the vaporization chamber. In some embodiments, two holders placed together in the vaporization chamber may include the same or different source materials. In some embodiments, a plurality of holders may be provided in a cartridge. The cartridge may be configured to be placed into inhaler 100, such that in each use a single holder may be, automatically placed in the vaporization chamber.

In some embodiments, the inhaler may include one or more vaporization chambers configured to accept two or more holders at a time. This may allow to provide to two or more source materials in the holders using the same or different operation profiles, and may deliver the combined vapor (or aerosol) in a single inhalation. In some embodiments, a single holder may include a plurality of individually addressable source materials, such that two or more source materials in the holders that may undergo the same or different operation profiles, and may deliver the combined vapor (or aerosol) in a single inhalation. In some embodiments, different source materials may be disposed in the same holder, at different sections of the holder (optionally independently heatable sections). Alternatively, different source materials may be mixed together.

In some embodiments, heating operator 110 may be configured to provide heat to one or more vaporization chambers in the inhaler. In some embodiments heating operator 110 may include a power source 112 for providing electrical power to a heating element 114 (e.g., a resistor, such as a copper wire(s)). In some embodiments, heating element 114 may be in contact with the source material, for example, when heating element 114 (e.g., one or more metal meshes or wires) is included in a chip. Optionally, heating element 114 is or includes a U-shaped mesh spanning both opposing sides of the chip, such that electricity can run between a first electrode in contact with one side of the chip to another electrode on the other side, thereby to bilaterally heat the source material. In such case power source 112 may be configured to be electrically connected to heating element 114 of the chip when the chip is inserted to the vaporization chamber in inhaler 110. In some embodiments, when more than one source materials are positioned in the chamber, each connected to a distinct heating element 114 of the chip, power source 112 may be configured to be electrically connected to each distinct heating element and to activate one or more of the heating elements.

Additionally, or alternatively, heating element 114 may be included in heating operator 110 and may come to be in contact with the source material, when the source material is inserted to the vaporization chamber in inhaler 100 or is moved, manually and/or automatically within inhaler 100 so as to come into such contact at the vaporization chamber. In some embodiments, the vaporization chamber may be or may include any cavity/holder//carrier/place that may be configured to hold the source material and/or to allow positioning of the source material in contact with a heating element or a heated airflow, therefore to cause the vaporization, aerosolization, volatilization, or any other form of extraction of two or more substances from the source material into a gashouse environment. The vaporization chamber may be configured to hold any form of source material, including one or more of a solid source material, a liquid source material, and/or a mixture of solid and liquid.

In some embodiments, heating element 114 may be included in heating operator 110 and may be configured to provide heat to airflow produced in or by airflow system 120. In some embodiment airflow in airflow system 120 is produced by the inhalation force of a user.

Additionally, or alternatively, airflow is produced by the inhaler itself, for example using a fan, ventilator, and/or any suitable type of air blower.

In some embodiments, heating element 114 may be located in the route of the airflow before the airflow meets the source material, such that the source material is subjected to an already heated airflow.

Accordingly, controlling heating operator 110 and/or airflow system 120 (e.g., by controller 130) may allow to provide a selected temperature-time profile to the vaporization chamber. For example, controller 130 may control the amount of power supplied by power source 112 to heating element 114, the duration at which the power is supplied and/or the timing for the application of power. Controller 130 may receive temperature measurements from a sensor located in proximity to the source material and may control heating operator 110 at least partially according to the sensor's readings (e.g. temperature measurements, impedance measurements, electrical resistance measurements and the like). For example, constant amount of power or a constant temperature may be applied to the source material for various durations (e.g., 0.5, 1, 2, 3, seconds, etc.) at one or more operation sequences (e.g., delivery of vapor or inhalation). In another example, different amounts of power and/or different temperatures may be applied for different or for the same periods of time during an operation sequence or inhalation, for example, by an instant increase in power/temperature from one level to the other and/or by a shutdown power delivery for a defined period of time. In yet another example, a continuous change (increase and/or decrease) in the power and/or temperature may be applied as a function of time.

In some embodiments, airflow system 120 may be configured to provide airflow to the one or more vaporization chambers. In some embodiment airflow in airflow system 120 is produced by the inhalation force of a user. Additionally, or alternatively, airflow is produced by the inhaler itself. Airflow system 120 may include any component/element that may provide a predetermined airflow-time profile to the one or more vaporization chambers. Airflow system 120 may include any number of pipes or conduits and one or more valves for directing airflow via the one or more vaporization chambers and/or though one or more alternative routes towards a vapor/aerosol exit (e.g., exit 122 illustrated in FIG. 1B). In some embodiments, airflow not carrying vapor/aerosol may flow through the vapor exit, for example via one or more bypass routes. The one or more valves may include fixed valves (e.g., for providing a fixed amount of air or fixed rate of airflow via at least a given conduit) or adjustable valves that may be adjusted (e.g., by adjusting a passageway and/or closing/opening a shutter). For example, such a valve may adjust an airflow-time profile caused by the inhalation of air (by a user) to be the same for all users regardless to the user (e.g., an adult or a child). In some embodiments, controlling the airflow may include a complete opening/closure of the valve (e.g., by controller 130). For example, controlling the opening and closure of the valve may be done according to reading/information received from a pressure sensor, a flow sensor (e.g., a flowmeter) and the like. Such readings may indicate how much sir is provided to the vaporization chamber. Accordingly, controller 130 may control the opening/closure of the valve to provide the selected airflow-time profile. In some embodiments, if an excess air was introduced into airflow system 120, a valve that opens a bypass route may be opened and at least some of the air may be directed through that valve.

In some embodiments, airflow system 120 may include a fan and a motor. In some embodiments, controller 130 may be configured to control the motor and or a gear included in airflow system 120 to provide a controller airflow to the vaporization chamber. In some embodiments, the fans of system 120 may be controlled based on information/reading from a sensor as disclosed above with respect to the valve.

Accordingly, in some embodiments, controlling airflow system 120 (e.g., by controller 130) may allow to provide a selected airflow-time profile to the vaporization chamber. For example, controller 130 may control the diameter of the passageway (e.g., by closing and/or opening a shutter) and the timing for the application of the airflow (e.g., the duration at which the shutter is at least partially open or the duration at which the fan is operated). In some embodiments, constant air capacity (and/or airflow rate) may be applied to the vaporization chamber for various durations (e.g., 0.5, 1, 2, 3, seconds, etc.) at each operation (e.g., delivery of vapor). In some embodiments, different air capacities or rates may be applied for different or the same periods of time, for example, by a discrete increase in the diameter of the valve's passageway or discrete increase in the velocity of the fan for a defined period of time. In some embodiments, a continuous change (increase and/or decrease) in the air capacity or rate may be applied as a function of time. In some cases airflow rate may increase multiple times and/or decrease multiple times during operation, to follow a given operation profile.

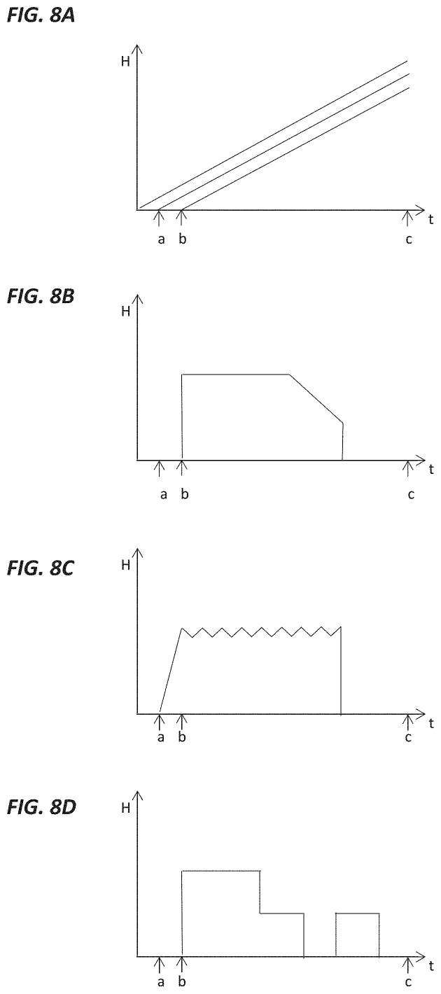

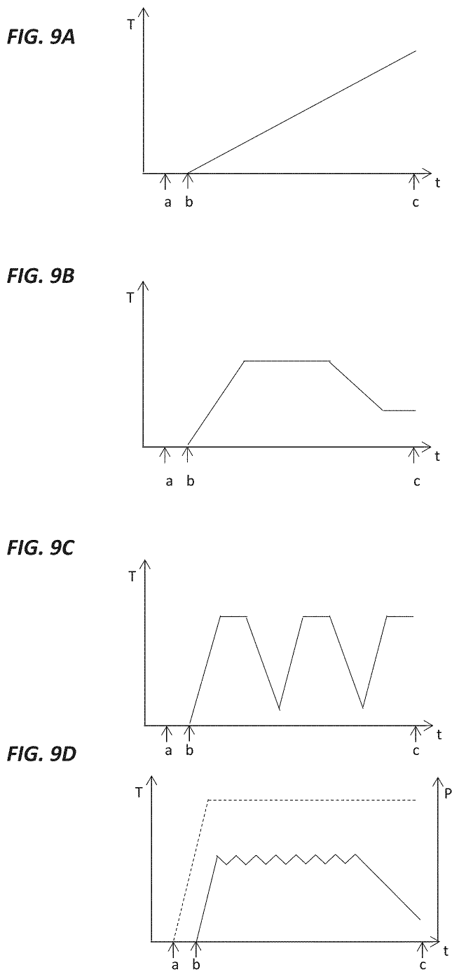

In some embodiments, an operation profile may include any combination of temperature-time profile and/or airflow-time profile. Accordingly, controller 130 may control power source 112 of heating operator 110 and airflow system 120 to provide any temperature-time profile and/or airflow-time profile. For example, controller 130 may control heating operator 110 and airflow system 120 to provide constant amount of power at a constant air capacity for different time durations. In another example, controller 130 may control heating operator 110 and airflow system 120 to provide a constant air capacity at two different amounts of heating power for the same or different amounts of time. Some example, of heating-time profiles and temperature-time profiles are given in FIGS. 8A-8D and 9A-9D.

FIGS. 8A-8D are graphical representations of heating-time profiles according to some embodiments of the invention. The heating H in each graph may present the power and/or the current supplied by power source 112 to heating elements (e.g., heating elements 114 illustrated in FIG. 1D). Accordingly, in FIG. 8A a linearly increasing of the amount of power/current may be provided, for example, to three different heating elements and the heating may be initiated at different times for each element. In some embodiments, the first heating element may be powered at the air-flow commence (at t=0) and the following two heating elements may be powered at later times (t=a and t=b). In FIG. 8B heating may start sometimes after the air-flow commence (at t=b) instantly increase to a constant high level for a predefined duration and then linearly decreased. In FIG. 8C heating may start at t=a, instantly increase to a certain level and then fluctuate between two power/current levels until being powered-off. In FIG. 8D heating may start sometimes after the air-flow commence (at t=b) instantly increase to a constant high level for a predefined duration followed by instant decrease to a constant level, total power shutdown and instant increase to a constant level.

FIGS. 9A-9D are graphical representations of temperature-time profiles according to some embodiments of the invention, received for example, from a temperature sensor located in proximity to the source material. The temperature time profiles may be affected by both the heating provided and the airflow provided to the source material. Graphs 9A-9D show various optional behaviors of the temperature, from linear increase (FIG. 9A), to linear increase until a plate followed by a linear decrease (FIG. 9B), various increasing and decreasing of the temperature (FIG. 9C) and fluctuating temperature profile (FIG. 9D).

Referring back to FIG. 1A, in some embodiments, controller 130 may include a processor 132, a memory 134 and a communication unit 136. Processor 132 may be, for example, a chip or any suitable computing or computational device. Memory 134 may include a double data rate (DDR) memory chip, a flash memory, a volatile memory, a non-volatile memory, a cache memory, or any other suitable memory unit or storage unit. Memory 134 may store any executable code, e.g., an application, a program, a process, task or script. The executable code may include instructions for controlling at least some of the components of an inhaler according to embodiments of the invention (e.g., airflow system 120 and heating operator 110) and/or any other codes or instruction for executing methods according to embodiments of the present invention. The executable code may be executed by processor 112.

In some embodiments, communication unit 136 may include any communication module configured to communicate wirelessly (and/or wired) with an external computing device, for example, an external server 10 or a user device 20. Sever 10 may be any computing and storing platform, for example, a cloud-based computing service and/or a cloud-based storage that is configured to communicate with controller 130. Server 10 may include data related to the operation of inhaler 100. For example, server 10 may store a plurality of operation profiles each being associated with the delivery of different amounts of one or more substances from a source material. User device 20, may be for example, a personal computer, a desktop computer, a mobile computer, a laptop computer, a notebook computer, a tablet, a smartphone, a smartwatch and the like.

In some embodiments, inhaler 100 may include a user interface 140 configured to receive/display information from/to a user. For example, user interface may include a screen (e.g., a touchscreen), one or more buttons (e.g., on/off button), one or more light sources (e.g., LED) a speaker and the like. User interface 140 may be configured to receive selection of parameters and/or display indications, as will be further disclosed.

Reference is now made to FIG. 1B which is an illustration of an inhaler 100 according to some embodiments of the invention. The design of inhaler 100 and any of the components of inhaler 100 illustrated in FIG. 1B are given as an example only and the invention, as a whole, is not limited to a particular design or a particular inhaler. Inhaler 100 may include a body 105 for holding at least some of the components of inhaler 100, for example, heating operator 110 (not illustrated), controller 130 (not illustrated) airflow system 120 (partially illustrated) and user interface 140. In some embodiments, at least a portion of airflow system 120 may be detachable from body 105 (as illustrated). Airflow system 120 of inhaler 100 may include any number of pipes or conduits and one or more valves (not illustrated) for directing the airflow via the pipes, for example via opening 125 towards vapor exit 122. In some embodiments, the source material may be located at a vaporization chamber being in proximity to, or attached to, opening 125 when both are in use position within body 105. In some embodiments, a chip 160 (illustrated in FIGS. 1C and 1D) may be in contact with the pipe at opening 125 such that air may flow through chip 160 into opening 125 towards vapor exit 122. In some embodiments, the vaporization chamber (not shown) may be a cavity/holder/carrier/place that may be configured to hold the source material directly or indirectly by holding a holder comprising the source material. The vaporization chamber may be configured to hold source material including a solid source material, a gel source material, a liquid source material and/or a mixture of solid, gel and/or liquid.

In some embodiments, user interface 140 may include a button 142. In some embodiments, user interface 140 may include a touchscreen or any other interface suitable for receiving input from a user of inhaler 100. In some embodiments, user interface 140 may configured to receive sound, for example voice commands.

Reference is now made to FIGS. 1C and 1D which are a side view and a top view of a chip for holding a source material according to some embodiments of the invention. In some embodiments, chip 160 may include a cavity 165 for accepting the source material. In some embodiments, chip 160 may include one or more heating elements 114 (for example, a conductive mesh, as illustrated in FIG. 1D) for providing heat to the source material in cavity 165. In some embodiments, heating elements 114 may be provided with electrical power from power source 112 (illustrated in FIG. 1A). In some embodiments, chip 160 may be attached to the vaporization chamber in airflow system 120 such that the airflow controlled by airflow system 120 (e.g. generated by an inhaling user) may flow through cavity 165 towards vapor exit 122.

In some embodiments, chip 160 may be configured to hold (e.g., in cavity 165) a source material that includes two or more different source materials (e.g., cannabis and a synthetic polymer carrying morphine). The two materials may be mixed together, may be placed in chip 160 side by side, one on top of the other, and the like. Accordingly, cavity 165 may include at least two separate sub-compartments or areas each for holding separate (optionally different) source materials and inhaler 100 may be configured to apply the same and/or different operation profiles simultaneously to all (or some) sub-compartments or area, or to each sub-compartment or area separately. According to some embodiments, two separate cavities, such as cavity 165 may be used, each adapted to receive and retain a source material or, for example, a double dose of the same source material or two different source materials (e.g. raw cannabis of different strains; or the same strain but having been grown under different growth conditions). It should be appreciated that any combination of dosage, combination of source materials etc., may be used. In some embodiments inhaler 100 may be configured to deliver in a single operation sequence (e.g. a single inhalation) substances from two or more chips 160, simultaneously and/or in sequence.

In some embodiments, inhaler 100 may be configured to deliver in a single inhalation at least two different substances at a predetermined ratio or predetermined amounts, from a defined source material. As used herein a defined source material may be any natural and/or synthetic source material that contains a substance to be delivered or a precursor thereof and at least one additional defined characteristic, for example, a known weight and/or an amount or concentration of the at least one substance or precursor thereof. For example, the defined source material may be cannabis that contains a known percentage or weight of total THC (as one or more of THCA and Δ9-THC).

Reference is now made to FIG. 2 which is a flowchart of a method of providing two or more substances according to some embodiments of the invention. In 210, a defined source material may be provided to an inhaler, for example, inhaler 100. The defined source material may be inserted to one or more vaporization chambers in inhaler 100. For example, a chip (e.g., chip 160) comprising a defined weight of a known type of tobacco may be inserted into inhaler 100. In another example, a chip that includes cannabis having a defined amount of THCA (e.g., 3 mg) may be inserted. In yet another example, a mixture of 10 mg of cannabis and 10 mg of a synthetic carrier comprising morphine may be placed inside inhaler 100. In yet another example, the defined source material may include a mixture of a botanical material and an extract (either from a botanical material or from synthetic source). The botanical material may carry the extract. In some embodiments, the botanical material (e.g. 20 mg of raw cannabis or raw tobacco) may include least one natural substance found within the botanical material (e.g. cannabis containing THC, Δ9-THC, and/or THCA or tobacco containing nicotine) and the extract may include a synthetic or extracted substance (e.g. morphine) carried by the botanical material. In some embodiments the extract comprises a substance that is found within the botanical material which carries the extract.

In some embodiments, the provision of the source material may be performed manually by a user or automatically by the inhaler, by adjusting the location or position of one or more components within the inhaler. For example, a user may directly place the defined source material (e.g., chopped cannabis) in the vaporization chamber or may place a holder (e.g., a chip, a capsule and the like) holding the source material in the vaporization chamber. In yet another example, inhaler 100 may bring the source material into the vaporization chamber for use by moving a conveyor (e.g., a ribbon or a belt) carrying the source material and/or by placing (e.g., using a lever) the source material directly or a chip or pod holding the source material. In some embodiments, the source material may be stationary, and the inhaler may adjust the position of the source material and/or opens a valve and/or selects an electric circuit to deliver electricity and/or direct airflow to the source material placed in the vaporization chamber.

In some embodiments, controller 130 may receive the definition of the source material form a user via a user interface or automatically from a reader for reading machine readable elements associated with inhaler 100, for example, an RFID reader to a barcode reader, when the source material is tagged with a reading machine readable element. In some embodiments inhaler 100 (and controller 130) may be configured to use only the same defined source material (or group of source materials) in each operation.

In some embodiments, the defined source material may be selected manually by the user, a physician a caregiver and the like. The user having two or more source materials may select to insert inhaler 100 a first type of source material of the plurality according to a recommendation given by the physician or a required affect known to be delivered by the first source material. In some embodiments, the defined source material may be selected automatically, for example, according to a predefined required effect. In some embodiments, controller 130 may select to use a first type of source material included in one or more cartridges having together at least two different types of chips holding different types of source materials. Controller 130 may select to automatically place at least one chip of a first type according to predetermined requirements, for example, a treatment protocol received from a physician, a required effect received from the user and the like.

In 220, in accordance with some embodiments, a selected operation profile may be applied to the defined source material. The selected operation profile may, for example, be designed to deliver from the defined source material a first substance and, for example, a second substance at a predetermined ratio associated with the selected operation profile. In some embodiments, changing the operation profile (e.g., selecting a different operation profile) may affect the ratio. For example, a plurality of operation profiles may be stored in memory 134 or server 10, each of the operation profiles in the plurality of operation profiles may be associated with a different ratio between the first and the second substances. In some embodiments, 220 is preceded with 215, wherein the operation profile is selected according to the predetermined ratio.

In some embodiments, controller 130 may be configured to receive a selected operation profile associated with the delivery of a first substance and a second substance from a defined source material at a predetermined ratio. Controller 130 may control heating operator 110 and/or airflow system 120 to provide the selected operation profile. A plurality of operation profiles or data indicative of these operation profiles may be stored in a database associated with controller 130 (e.g., memory 134 or a cloud-based database included in server computer 10). The database may include lookup tables for associating various operation profiles with different ratios, formulas that calculate the temperature-time profile and/or an airflow-time profile required to produce specific ratios and the like. In some embodiments, each operation profile or predetermined ratio may be associated with an indication to be presented to a user. In some embodiments, the operation profiles may be associated with an expected effect on the user, and the selection is based on an expected effect and/or relative to the user's (or other's) past experience, without direct reference to specific source material(s), substance(s), profile(s) and/or ratios.

Some examples for operation profiles for delivery of Δ9-THC and THCA from cannabis are given herein. For example, 3 different operation profiles may all include heating a source material that include 15 mg cannabis to a temperature of 180° C. The three operation profiles may vary in the duration during which heat is provided to the source material, for example, heating for 1 second may result in receiving a first Δ9-THC:THCA ratio, heating for 2 seconds may result in a second Δ9-THC:THCA ratio and heating for 3 seconds may result in a third ratio Δ9-THC:THCA.

In yet another example, the time-temperature profile and the airflow-time-profile may vary between different operation profiles, however all the operation profiles may result with the same ratio of Δ9-THC:THCA (e.g. 1:1). A difference between the aerosols resulting from such operation profiles may manifest in the total amount of Δ9-THC&THCA and/or in the amount of a third substance.

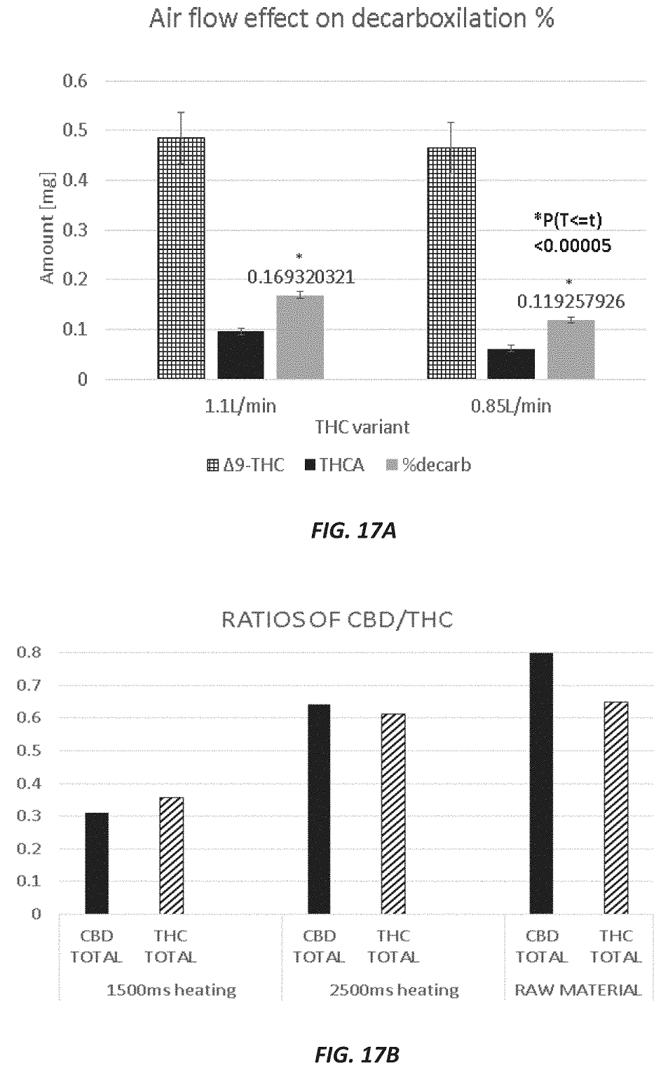

In the following experiment performed by the inventors, the effect of airflow rate on decarboxylation of THCA was tested. 14 samples (7 samples tested in each of the profiles) of 13.5 mg each of Bedrocan strain (Bedrocan, Holland) were heated for 1620 ms to a temperature of 185° C. In a first operation profile (upper table), the rate of flow through the sample was set to 1.1 L/min; in a second operation profile (lower table), the rate of airflow through the sample was set to 0.85 L/min. Aerosol was collected and extracted and analyzed in UPLC (Ultra performance liquid chromatography) for the presence of THCA and Δ9-THC. As can be observed, in this example, under certain conditions, increasing the rate of flow through the sample increased the percentage of THCA in the aerosol. (As shown below, the percentage of THCA was calculated as the amount of THCA divided by the total amount of both THCA and Δ9-THC.) The graph of FIG. 17A presents the results shown in the table below.

TABLE 1

| | | | | | % THCA/ |
|---|---|---|---|---|---|
| | | | Δ9- | | TOTAL |
| Constant | | flow | THC | THCA | (Δ9-THC + |
| parameters | Sample | rate | (mg) | (mg) | THCA) |
| 185° C., | 1 | 1.1 | 0.527 | 0.099 | 16% |
| 1620 ms | 2 | L/min | 0.431 | 0.091 | 18% |
| | 3 | | 0.587 | 0.110 | 16% |
| | 4 | | 0.462 | 0.095 | 17% |
| | 5 | | 0.452 | 0.092 | 17% |
| | 6 | | 0.445 | 0.091 | 17% |
| | 7 | | 0.484 | 0.093 | 16% |
| | SD | | 0.051213244 | 0.006278479 | 0.006292045 |
| 185° C., | 8 | 0.85 | 0.521 | 0.072 | 12% |
| 1620 ms | 9 | L/min | 0.559 | 0.076 | 12% |
| | 10 | | 0.529 | 0.058 | 10% |
| | 11 | | 0.486 | 0.066 | 12% |
| | 12 | | 0.434 | 0.056 | 12% |
| | 13 | | 0.357 | 0.054 | 13% |
| | 14 | | 0.365 | 0.049 | 12% |
| | SD | | 0.074587999 | 0.009311198 | 0.009506462 |

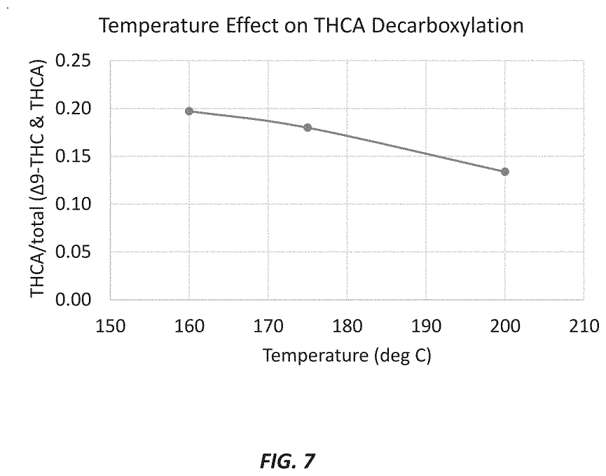

In another experiment performed by the inventors, various operation profiles were tested. In lines 1-9 of the table below, a change in the heating temperature was tested, while the rate of airflow and the heating duration remained constant. Aerosol was collected and extracted and analyzed in UPLC for the presence of THCA and Δ9-THC. A graph presenting the results obtained for the various heating temperatures is shown in FIG. 7 and further discussed below; in lines 10-14 of the table, a change in the rate of airflow was tested, while the temperature and the heating duration remained constant; in lines 17-19 of the table, a change in the heating duration was tested, while the temperature and rate of airflow remained constant. As can be observed, in this example, under certain conditions, increasing the heating temperature (while maintaining other parameters (flow rate, duration of heating) constant) reduced the percentage of THCA released; lengthening the heating duration (while maintaining other parameters constant) reduced the percentage of THCA released. (As shown below, the percentage of THCA was calculated as the amount of THCA divided by the total amount of both THCA and Δ9-THC.)

TABLE 2

| | | | | | % THCA/ |
|---|---|---|---|---|---|
| | | | Δ9- | | TOTAL |
| constant | | parameter | THC | THCA | (Δ9-THC + |
| parameters | Sample | changed | (mg) | (mg) | THCA) |
| 1.1 | 1 | 160° C. | 0.220 | 0.054 | 20% |
| L/min, | 2 | | 0.201 | 0.044 | 19% |
| 1500 ms | 3 | | 0.208 | 0.051 | 20% |
| | 4 | 175° C. | 0.475 | 0.101 | 18% |
| | 5 | | 0.461 | 0.101 | 18% |
| | 6 | | 0.478 | 0.100 | 18% |
| | 7 | 200° C. | 0.972 | 0.146 | 13% |
| | 8 | | 0.831 | 0.131 | 14% |
| | 9 | | 0.790 | 0.117 | 13% |
| 175 C., | 10 | flow 1.2 | 0.467 | 0.109 | 19% |
| 1500 ms | 11 | L/min | 0.423 | 0.098 | 19% |
| | 12 | flow 1.3 | 0.459 | 0.116 | 21% |
| | 13 | L/min | 0.468 | 0.104 | 19% |
| | 14 | | 0.440 | 0.095 | 18% |
| 185° C., | 15 | 600 ms | 0.093 | 0.026 | 22% |
| 1.1 | 16 | 800 ms | 0.315 | 0.069 | 18% |
| L/min | 17 | 1000 ms | 0.620 | 0.103 | 14% |

Returning to FIG. 2, in some embodiments, the selection (e.g. of amounts, ratios, and/or an operation profile) may be received from user interface 140 and/or user device 20. For example, optional operation profiles or indicators associated with different operation profiles may be presented on the screen of user interface 140 or on the screen of user device 20 associated with the user, and the user (e.g., a patient, a caretaker, a physician and the like) may select a required operation profile. In some embodiments, the operation profiles may be preselected by the user (or for the user, for example, by a physician) according to a treatment regimen or for pure recreational purposes. Additionally, or alternatively, optional ratios/amounts or indicators associated with different ratios/amounts may be presented. In some embodiments, indicators associated with different operation profiles may include treatment parameters (e.g., levels of pain, decrease pain, increase breathing and the like) and/or sensory choices (e.g. smell preferences, sweetness, etc.). Additionally, or alternatively, the indicators may refer to side effects (e.g., increase/decrease drowsiness, decrease nausea and the like). In some embodiments, the indicators may be related to predetermined relationships between treatment parameters and side effects, for example, decrease pain but increase nausea or vice versa). In some embodiments, the indicators may be related to past experience, for example, choose the one selected two days ago, this morning etc.

In some embodiments, controller 130 may be configured to receive at least one signal indicative of a condition of a user taking the first and second substances. In some embodiments, at least one signal may be received from the user (e.g., via user interface 140 and/or user device 20), for example, an indication regrading a general feeling of the user (e.g., the level of pain in a scale of 1-10) and/or an indication from the user regarding general satisfaction or satisfaction with one or more sensory effects (for example too sweet smelling or not enough lemony). In some embodiments, the signal may include a user request, for example, a request for another dose (due to pain, improve breathing difficulties (following the use of Salbutamol) or as a general desire of the user, or any recreational preference. In some embodiments, the signal may be a feedback signal indicative of an effect of at least one previous inhalation. For example, the signal may be received from the user (e.g., via user interface 140 and/or user device 20) stating his/her feeling before the inhalation and/or following the inhalation. Additionally, or alternatively, the signal may be received automatically from an external medical device, for example, a smartwatch measuring the user's heartrate or a sphygmomanometer measuring the user's blood pressure and the like. The feedback signal may give an indication to the medical condition of the user and/or the physical effect of the inhalation on the user, for example, reductions in heartrate and/or blood pressure. In some embodiments, the operation profile may be selected (e.g., by controller 130) based on the received signal. Optionally, operation profiles are automatically selected, or are restricted to a subset of operation profiles optionally based on received input.

In some embodiments, a set of operation profiles may be received (e.g., by controller 130). In some embodiments, this may be a selected subset of the plurality of operation profiles. In some embodiments, each one of the operation profiles set may be configured to deliver the same predetermined amount of the first substance. For example, all the operation profiles in the selected subset may be configured to deliver a fixed amount (e.g. 250 μg) of CBD and different amounts of THC (or Δ9-THC) or vice versa.

In some embodiments, the operation profile may be selected to controllably cause a production of the first substance from at least a portion of the second substance. For example, the operation profile may be selected to control a rate of decarboxylation of at least a portion of THCA or CBDA into Δ9-THC or CBD respectively. In some embodiments, the operation profile may be selected to control a rate of decarboxylation of at least a portion of CBCA into CBC, THCVA into THCV, and/or CBGA into CBG.

Attention is now redrawn to FIG. 1A. In some embodiments, a selection of the operation profile may be conducted by server computer 10. Server 10 may include a database for storing thereon data indicative of a plurality of operation profiles (as disclosed above), such that each of the operation profiles is designed to deliver from a source material a first substance and a second substance at a predetermined ratio and/or predetermined amounts. In some embodiments, each operation profile comprises at least one of: a temperature-time profile and an airflow-time profile. Optionally, an operation profile includes spatial definition. In some embodiments, server 10 may further include a processor (e.g., a cloud based processing unit) that may be configured to receive from a first remote device (e.g., inhaler 100, user device 20 and the like) data indicative of a predetermined ratio between the first and the second substances, for example, an indicator (e.g., a number), data related to a general feeling of the user, data related to the medical condition of the user and the like. In some embodiments, server 10 may further select an operation profile from the stored plurality of operation profiles that is designed to provide the predetermined ratio or improve the general feeling and/or medical condition of the user. In some embodiments, server 10 may randomly choose the first operation profile for the first inhalation, and may adjust (e.g., change) the selection after receiving a signal indicative of the user's condition following the first inhalation. Server 10 may send the selection to controller 130.

In some embodiments, controller 130 may be configured to cause the inhaler (e.g., inhaler 100) to apply the selected operation profile to the defined source material when located in the inhaler. Controller 130 may control at least one of: the heating operator (e.g., heating operator 110), the airflow system (e.g., airflow system 120) and a spatial distribution thereof to apply the selected operation profile to the defined source material when located in the vaporization chamber in the inhaler. For example, controller 130 may control the power supplied to power source 112, the duration and timing of the application of the power, one or more valves and/or one or more fans included in airflow system 120, a rate of airflow through the source material and the like. In some embodiments, the selected operation profile may include any combination of temperature-time profile and/or airflow-time profile and may be configured to deliver from the defined source material the first substance and the second substance.

In some embodiments, inhaler 100 may be configured to apply each of a plurality of operation profiles to a defined source material, such that each operation profile is designed to deliver a predetermined amount of a first substance and different amounts of one or more other substances. In some embodiments, inhalation of the first substance may be required to achieve a first effect on the user (e.g., pain relief) and the one or more other substances may have at least one second effect, for example, reduce nausea or reduce an undesired side effect (e.g. a cognitive state altering effect). In some embodiments, the amount to be delivered of the first substance may be set or fixed (e.g., due to potential adverse effects) however, the one or more other substances may be delivered under less stringent constraints. In some embodiments, inhalation of the first substance may be for recreational or addiction related purposes (for example, reach a high with THC or inhale nicotine to satisfy and urge) and the one or more other substances may have at least one second effect, for example, a pleasing aroma or other desired effect. When used for recreational purposes, it may be desired to maintain the amount of the first substance low in each operation profile, thereby to increase the number of inhalations required to achieve its desired effect.

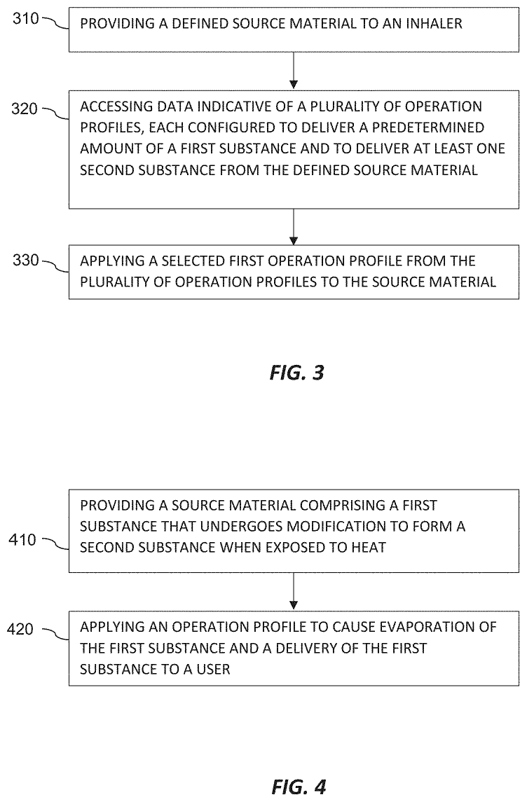

Reference is now made to FIG. 3 which is a flowchart of a method of providing substances according to some embodiments of the invention. In 310 a defined source material may be provided in an inhaler, for example, inhaler 100. The defined source material may be inserted to one or more vaporization chambers in inhaler 100. For example, a chip (e.g., chip 160) comprising a defined weight of a known type of tobacco may be inserted into the vaporization chamber of inhaler 100. In another example, a chip that includes cannabis having a defined amount of THCA may be inserted. In yet another example, a mixture of 0.5 gr of cannabis and 0.5 ml of morphine may be placed inside inhaler 100. In yet another example two or more synthetic and/or isolated active substances are provided in association with a carrier pallet (e.g. cellulose and/or a synthetic polymer).

In 320, according to some embodiments, data indicative of a plurality of operation profiles may be accessed. Each operation profile from the plurality of operation profiles may be configured to deliver from the defined source material the same predetermined amount of a first substance and to deliver at least one second substance from the defined source material, and the plurality of operation profiles may each cause the delivery of a different amount the second substance. For example, all the operation profiles in the plurality may be configured to cause the delivery of 0.25 mg THC (being a sum of Δ9-THC and THCA or just Δ9-THC). However, each of the operation profiles may be configured to deliver different amounts of one or more of THCA, CBD, CBDA, a terpene and the like. In some embodiments, the plurality of operation profiles may vary in at least one of: a temperature-time profile, an airflow-time profile and spatial definition as disclosed above with respect to inhaler 100 and/or the method of FIG. 2.

In some embodiments, the plurality of operation profiles may be stored in a database, for example, memory 134 and/or the database of server 10 and may be accessed by a processor, for example, processor 132 of controller 130 and/or sever 10.

In some embodiments, the defined source material may include cannabis and the first and/or second substance may be selected from a group consisting of: Δ9-THC, THCA, CBD, CBDA, Cannabinol (CBN), Cannabigerol (CBG), Cannabichromene (CBC), Cannabicyclol (CBL), Cannabivarin (CBV), Tetrahydrocannabivarin (THCV), Cannabichromevarin (CBCV), Cannabigerovarin (CBGV), Cannabigerol Monomethyl Ethe (CB GM), Cannabielsoin (CBE) and Cannabicitran (CBT), as well as one or more terpenes and/or flavonoids present in cannabis. Tobacco may include for example syzygium aromaticum and menthol.

In some embodiments, controller 130 may receive a selection of a first operation profile from the plurality of operation profiles listed above. In some embodiments, the selection may be received from at least one of: user interface 140, user device 20 and server 10. In some embodiments, the plurality of operation profiles may further vary from one another in the effect, on the user, of giving the two or more source materials by applying the profile. Accordingly, a user (e.g., a patient or a professional) may select the operation profile according to a required effect. For example, a first profile may cause the provision of an aerosol that reduces pain and nausea and a second profile may cause the provision of an aerosol that is more prone to reduce pain and improve the appetite. In some embodiments the operation profiles provide aerosols each of which strikes a different balance between two effects (e.g. pain relief and dizziness) allowing the user to select a desired balance, based on preference and/or circumstances (such as before going to bed as compared with going to work). In some embodiments, an application running on a personal device (e.g. a user's smartphone) may present the two or more options to be selected by the user.

In some embodiments, the selection of the operation profile may be based on a received first signal as discussed above with respected to the method of FIG. 2. The first signal may be received from the user, via user device 20 or user interface 140 or from an external device. In some embodiments, controller 130 and/or server 10 may select the operation profile based on the received signal.

In some embodiments, at least some of the operation profiles in the plurality of operation profiles may be configured to deliver a first predetermined amount of a first substance, a second predetermined amount of a second substance and at least one third substance from the source material. In some embodiments, the at least some of the operation profiles may vary in the amount of the at least one third substance. For example, the operation profiles may all be configured to apply predetermined amounts of total THC (or of Δ9-THC) and morphine and may vary in the amounts of THCA, CBD and/or CBDA.

In 330, the selected first operation profile from the plurality of operation profiles may be applied to the source material. In some embodiments, controller 130 may activate inhaler 100 to apply the selected operation profile to the defined source material when located in the inhaler. Controller 130 may control at least one of: heating operator 110 and airflow system 120 to provide the selected first operation profile from the plurality of operation profiles to the source material located at a vaporization chamber in inhaler 100.

In some embodiments, after the application of the first operation profile and the provision of the vapor a second signal may be received. The second signal may be received from the user, via user device 20 or user interface 140 or from an external device (e.g., a medical instrument, for example, a blood pressure or heartrate measuring devices), as disclosed above. In some embodiments, the selection of a second operation profile may be based on the received second signal. The selection may be done manually (e.g., a user selects from a list) or automatically, based on a defined protocol, by controller 130 and/or server 10 or by any other suitable controller. For example, if the heartrate measured after a first inhalation is above a predefined level but below another level, a second operation profile will automatically commence.

Some embodiments of the invention may be directed to method of providing two substances by inhalation such that one substance undergo heat generated modification from the other substance, for example, decarboxylation of THCA or CBDA to Δ9-THC or CBD, respectively. Upon exposing a source material, such as, cannabis to heat the THCA and CBDA tend to decarboxylate to Δ9-THC and CBD. Some embodiments of the present invention may allow a controlled decarboxylation process (e.g. by controlling the amount of heat provided to the source material and/or by controlling the rate of evacuation of the released substances by controlling airflow) thus controlling the presence of both Δ9-THC and THCA and/or CBD and CBDA at the same inhalation.

Reference is now made to FIG. 4 which is a flowchart of a method of providing two or more substances by inhalation. In 410, a source material is provided. The source material may include a first substance that undergoes modification to form a second substance when exposed to heat. The source material may be any source material according to embodiments of the invention. For example, the source material may include at least one of: a botanical material (e.g., cannabis, tobacco, fungus, etc.; in some embodiments the botanical material is or includes raw botanical material) two or more different botanical materials (e.g., cannabis and a fungus or two different strains or batches of cannabis), a mixture of two or more different botanical materials, a synthetic material, a synthetic material and a botanical material and a mixture of a synthetic material and a botanical material.

In some embodiments, at least one component in the source material (e.g., the cannabis) may include a first substance (e.g., THCA or CBDA) that undergoes modification (e.g., decarboxylation) to form a second substance (e.g., Δ9-THC or CBD) when exposed to heat. In some embodiments, the source material may be placed in an inhaler, such as, inhaler 100 as disclosed above. For example, an amount of the source material may be placed directly at a vaporization chamber in the inhaler. In another example, an amount of source material may be placed in a chip (e.g., chip 160) to be inserted to the vaporization chamber in the inhaler, the chip may either be included in a cartridge or not.

In 420, in accordance with some embodiments, an operation profile may be provided to cause vaporization of the first substance and a delivery the first substance to a user. In some embodiments, release of the first substance (e.g. by vaporization) is controlled to deliver a predetermined amount of the first substance.

Accordingly, the operation profile may be selected to allow only a partial modification of the first substance to the second substance. In some embodiments, the operation profile may cause a delivery of a predetermined amount of the second substance (e.g., Δ9-THC or CBD) together with the first substance (e.g., THCA or CBDA). In some embodiments, the operation profile may include at least one of: a temperature-time profile, an airflow-time profile and a spatial definition thereof, as discloses above with respect to operations 220 and 330 of FIGS. 2 and 3.

In some embodiments, controller 130 of inhaler 100 may control at least one of: heating operator 110 and airflow system 120 to apply the operation profile to a source material when located in a vaporization chamber (e.g., location 125) in inhaler 100. The operation profile may be designed to deliver from a defined source material a first substance and a predetermined amount of a second substance, the first substance may undergo modification to form the second substance when exposed to heat.

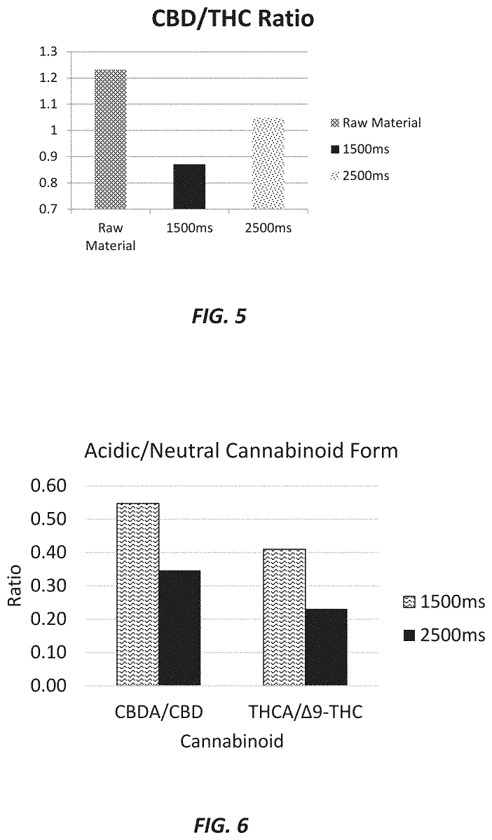

Reference is now made to FIG. 5 which is a bar chart of ratios between CBD/total THC received at various operation profiles according to some embodiments of the invention. Samples having 13.5 mg of a Bediol cannabis (Bedrocan. Holland) were tested at two operation profiles and compared to a raw material. The resulting aerosols was collected, extracted and analyzed by UPLC. The right bar shows a ratio of approximately 1.22 between the total cannabidiols (CBDA and CBDs) and the total tetrahydrocannabinols (THCA and Δ9-THC) in raw cannabis flowers. After heating to about a temperature of 180° C. under an airflow of 1 liter/minute for 1.5 seconds the ratio in the produced vapor dropped to approximately 0.87 meaning that the amount of total THC in the vapor is higher than the amount of CBD. Heating to the same temperature under the same airflow for 2.5 second changed the ratio in the produced vapor to almost 1 (50:50) CBD/THC. Accordingly, it has been established that changing the operation profile can controllably change the ratio between CBD/THC (or Δ9-THC) provided to a user in a single inhalation. Moreover, by applying one of the two operation profiles to each of a plurality of portions of the same source material, many in-between ratios are obtainable, depending for example of the relative amounts of source material exposed to each operation profile.

In the following related experiment performed by the inventors, samples having 13.5 mg of Bediol strain Cannabis (Bedrocan, Holland) were tested using two operation profiles and compared to a raw material. The samples were heated to a temperature of 180° C. under an airflow rate of 1.1 liter/minute. In the first operation profile, the heating duration was 1.5 seconds; in the second operation profile, the heating duration was 2.5 seconds. Aerosol was collected, extracted and analyzed using UPLC. As can be observed from this example, under certain conditions, lengthening the heating duration increased the CBD/total THC ratio (from about 0.79 in the first operation profile to about 0.96 in the second operation profile). As can also be observed from this example, under certain conditions, lengthening the heating duration released higher amounts of all active substances tested (CBDA, CBD, THCA, Δ9-THC). (API-active pharmaceutical ingredient). The graph of FIG. 17B presents the results shown in the table below.

TABLE 3

| | | | | total |
|---|---|---|---|---|
| constant | Heating | cannabinoid | mg | (acid + neutral |
| parameters | duration | API | API | form) |
| 180 C. | 1500 ms | CBDA | 0.125 | 0.311 |
| 1.1 L/min | | CBD | 0.201 | |
| | | THCA | 0.118 | 0.357 |
| | | Δ9-THC | 0.254 | |
| 180 C. | 2500 ms | CBDA | 0.188 | 0.642 |
| 1.1 L/min | | CBD | 0.477 | |
| | | THCA | 0.131 | 0.613 |
| | | Δ9-THC | 0.498 | |

Reference is now made to FIG. 6, which is a bar chart of the ratios between CBDA/CBD and THCA/Δ9-THC received at various operation profiles according to some embodiments of the invention. Samples having 13.5 mg of a defined type of cannabis were tested at two operation profiles. The samples were heated to a temperature of 180° C. under an airflow of 1 liter/minute) for 1.5 seconds and 2.5 second. Aerosol was collected, extracted and analyzed using UPLC. As can be seen from FIG. 6 the longer the operation profile the less amount of acids (THCA and CBDA) left after vaporization. In other words, a longer period of heating resulted in a higher amount of decarboxylation, which in turn resulted in higher amounts of Δ9-THC and CBD.

Reference is now made to FIG. 7 which is a graph showing the effect of the temperature on the relative amount of THCA vaporized. The experiment procedure and the results are also discussed above and presented in Table 2 Samples having 13.5 mg of a defined type of cannabis were tested by applying air at 1.1 liter/minute for 1.5 seconds at various temperatures from 160° C. to 200° C. The relative amount of THCA were measured against the total amounts of Δ9-THC+THCA. As can clearly be seen the higher the temperature the lower is the relative amount of THCA found in the aerosol. Accordingly, by selecting the temperature and/or the time-temperature profile, the amount of THCA provided during inhalation may be controlled (e.g., selected by the user).

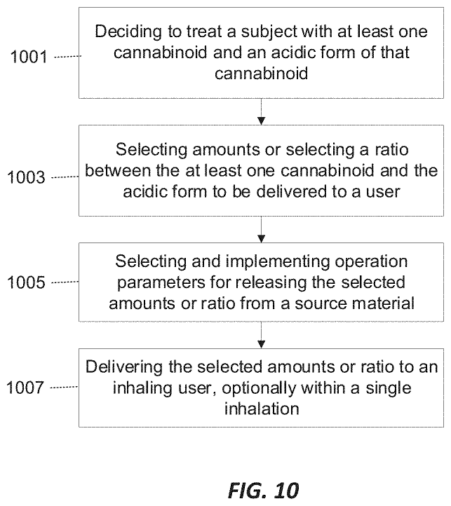

Reference is now made to FIG. 10, which is a flowchart of a method for delivering selected amounts and/or a selected ratio of at least one neutral cannabinoid and an acidic form of that cannabinoid to an inhaling user, according to some embodiments.

In some embodiments, a decision is made to treat a subject with at least one neutral cannabinoid and an acidic form of that cannabinoid (1001), for example, with Δ9-THC and THCA, CBD and CBDA, and/or others (e.g. CBDV and CBDVA, THCV and THCVA).

In some embodiments, a physician may decide to treat a user with THCA for addressing one or more of: pain, nausea, vomiting, inflammation, loss of appetite, ischemic/hypoxic injury, re-perfusion injury, anxiety, sleep disorders.

In some embodiments, amounts or a ratio between the at least one neutral cannabinoid and the acidic form of that cannabinoid are selected (1003). Optionally, the amounts or ratio are selected to produce a desired therapeutic effect and/or a psychoactive effect on the user. Optionally, the amounts or ratio are selected to produce a selected concentration of the provided substances in the blood. In some cases, blood concentration levels are built up as a cumulative effect of multiple deliveries, for example a plurality of inhalations and/or a plurality of use sessions (each use session including a plurality of inhalations), optionally set to be delivered over time. Optionally, only the amount of one of the neutral cannabinoid and the acidic cannabinoid or the total amount of the two is determined, but the ratios are different.

In some embodiments, selecting of amounts comprises setting a predefined, optionally constant amount of a substance. In an example, an amount of a neutral cannabinoid such as Δ9-THC is selected to be constant for a selected number of deliveries, and/or during a predefined time period during which the user uses the inhaler (for example, several hours, several days). In another example a total amount of a cannabinoid (e.g. Δ9-THC and THC taken together) is selected to be constant for a selected number of deliveries, and/or during a predefined time period during which the user uses the inhaler (for example, several hours, several days).

In some embodiments, while a first substance (e.g. a cannabinoid) is delivered at a pre-defined amount, an amount of a second substance such as the acidic form of that cannabinoid may be delivered in various and/or varying amounts, along with the first substance. In some cases, delivering of a first substance in constant, pre-defined amounts along with at least a second substance which varies in amount, for example varies for different inhalations and/or for different use sessions, leads to a different effect on the user. By modulating the amounts of at least a second substance and/or by modulating a ratio between the first substance which is provided at a predefined amount and at least the second substance, different blood concentrations of these substances may be reached, generating different physiological and/or cognitive effects on the user.

In some embodiments, an inhaler operation profile which defines a set of operation parameters is selected and implemented for releasing the selected amounts or ratios (1005).

Operation parameters may include, for example: a heating duration, a heating temperature profile, a duration of airflow, a rate of airflow, an extent of overlap between the passing of airflow and heating, a spatial definition of any of the aforementioned, and/or other parameters which are controllable and modifiable. Control of operation parameters may be carried out by the inhaler controller, by the user interface, by a remote server, and/or other. Physical modification of the operation parameters may be carried out by: adjusting a power applied for heating (e.g. adjusting an electrical current); adjusting airflow (e.g. via one or more valves within the inhaler); adjusting a sensor threshold (e.g. adjusting a temperature threshold above which heating is terminated; adjusting an airflow sensor threshold); adjusting a sensor position; adjusting a conduit passage or position; adjusting a chip position; selecting a different operation profile to one or more portions of the source material than to others.

In some embodiments, each operation profile generates release of substances at a composition different than a composition generated by another operation profile. In some embodiments, each operation profile generates release of two or more substances at a proportion different than a proportion generated by another operation profile.

In some embodiments, the released substances are delivered to the inhaling user (1007), optionally within a single inhalation of the user from the inhaler. In some embodiments, substance release begins only after the user begins inhalation.

In some embodiments, by selecting and/or modifying operation profiles, an entourage effect on the user may be affected. Optionally, blood concentration levels of the substances vary for each of the different profiles.

In some embodiments, two or more operations profiles are applied. In some embodiments, operation profiles are applied concomitantly to different source material units or source material portions in the same or different units, optionally even during a single inhalation of the user. In some embodiments, operation profiles are applied simultaneously, for example, using an inhaler which is configured to independently address and release substances from physically separated source materials. In an example, the inhaler is structured to receive two or more chips, optionally including two or more vaporization chambers and a separate flow path for each. In some embodiments, a different operation profile is then applied to each chip.

In another example, a chip, which is divided into a plurality of independently addressable regions is provided, the regions being separated, for example by an air seal and/or heat resistant barrier. Optionally, each of the regions comprises a different source material or mixture thereof. In some embodiments, a different operation profile is applied to each region of the chip.

In some embodiments, a total amount of a substance (e.g. of Δ9-THC) for delivering to the user is delivered over multiple inhalations and/or over multiple use sessions. In an example, a total amount of 1 mg Δ9-THC may be delivered in two separate sub amounts, comprising 0.5 mg each. In another example, 750 μg Δ9-THC may be delivered in two separate sub amounts, for example one including 500 μg and the other 250 μg. In yet another example, a 1 mg amount of total THC may be delivered from the same source material cannabis in each of (a) 1 inhalation of 1,000 μg THC, (b) 2 inhalations of 500 μg THC, (c) an inhalation of 500 μg THC and 2 inhalations of 250 μg THC or (d) 4 inhalations of 250 μg. Assuming that each inhaled amount (250, 500 and 1,000 μg THC) is associated with a different entourage, the combined entourages would be different for each of the alternative schemes.

In some embodiments, each sub amount of the selected substance is delivered using a different operation profile, thereby potentially releasing a different composition of substances that are released along with the selected substance. In an example, the 0.5 mg of Δ9-THC is released along with one or more substances such as THCA, terpenes, flavonoids, alkaloid, and/other cannabinoids, where the specific substance composition changes in response to changing the operation profile. Optionally, using this delivery scheme, delivery of the sub amounts of the selected substance will potentially generate a different entourage effect on the user, as compared to a single delivery of the large dose, and/or as compared to delivery of two smaller doses using the same operation profile.

In some embodiments, selection of an operation profile is performed by a user, for example according to one or more of: personal preferences, activities the user is planning to engage in (e.g. driving, in which it may be preferred to reduce a psychoactive effect, or sleeping, in which case it may be preferred to increase pain relief at the expense of generating dizziness or drowsiness); a current mental and/or physiological condition. Additionally, or alternatively, the inhaler or an associated device automatically recommends (e.g. the inhaler controller and/or an app on a user's mobile phone, optionally via the user interface) or directly applies one or more selected operation profiles. Selection of profiles may be performed in response to feedback provided by the user and/or obtained from the user, for example via the user interface and/or via one or more sensors.

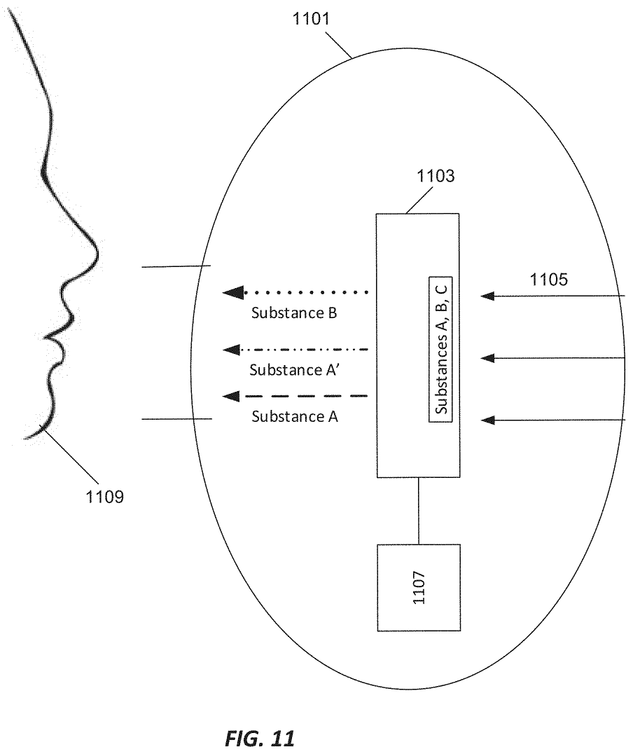

FIG. 11 schematically illustrates an inhaler for providing one or more substances released from source material to a user, according to some embodiments. In some embodiments, inhaler 1101 comprises or is configured to receive a source material chip 1103, including one or more substances (represented by A, B, C). In some embodiments, two or more substances of A, B, C are found within the same source material. Optionally, chip 1103 includes between 1-10 different source materials, for example a single source material, two different source materials, three different source materials.

In some embodiments, one or more substances (represented herein by A, A′, B) are releasable from the one or more source materials. In the example shown, substances A and B are released without being chemically modified; substance A′ represents a modification of substance A, for example, a chemical derivative or chemical modification of substance A. In an example, substance A is a substance which was naturally synthesized in the source material, and substance A′ is a chemical derivative of that substance (e.g. Δ9-THC, equivalent to A′ is a product of decarboxylation of THCA (equivalent to A), a naturally synthesized substance).

As further shown in this example, one or more substances within the chip such as substance C are not released from the chip. Substance C may include one or more of a carrier material; a substrate; a chemical additive, an inactive material (optionally an inactive material which naturally exists in the source material.) Optionally, substance C is added to the chip to accelerate or instead delay release of one or more other substances, for example by affecting a vaporization state, e.g. a vaporization temperature, of the one or more substances. In an example, glycerin is used, for example 1-5% glycerin is added to the source material.

In some embodiments, the one or more source materials in the chip are mixed together. Additionally, or alternatively, the one or more source materials are disposed inside the chip in manner in which different source materials are separated, for example, by one or more barriers configured in the chip. In an example, the chip is divided into a plurality of different regions, each region comprising a different source material. Optionally, in such construction, releasing of one or more substances from the chip involves the selection of one or more different source material regions.

In an example of substance release, flow of air 1105 is allowed or directed through the source material chip, passing through the source material, for example, in between particles of the source material.

In some embodiments, heating is applied to the substances in the chip. Heating may be applied simultaneously or at least partially overlapping with the passing of airflow through. Optionally, heating is applied only after airflow through the chip reaches a given threshold and remains constant. In some embodiments, heating is applied by one or more heating elements 1107, configured in the chip and/or in the inhaler. In an example, the chip comprises a heating element, for example in the form of a resistive mesh, and heating energy is applied to the mesh by one or more current conducting electrodes configured in the inhaler.

In some embodiments, an amount of at least one of the released substances A, A and/or B and/or a ratio between substance pairs are controlled. Optionally, inhaler 1101 is configured to operate according to a plurality of operation profiles, each operation profile defining a set of parameters to be applied, the parameters including, for example: a heating profile, an airflow profile, and/or a spatial definition thereof. In some embodiments, by selection of an operation profile, the user 1109 is provided with the selected amounts and/or ratios.

In some embodiments, the released substances (e.g. two or more substances) are released and delivered to the user during a single inhalation.

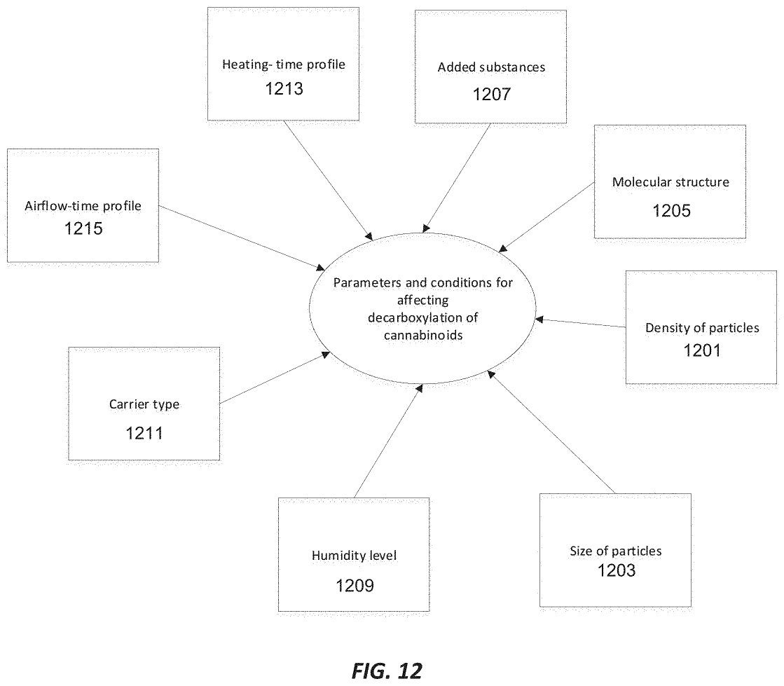

FIG. 12 is a diagram of parameters and conditions which affect decarboxylation of cannabinoids, according to some embodiments.

The following is a list including operational and/or structural parameters and conditions, which may be modified for affecting a chemical modification of one or more substances, such as decarboxylation of cannabinoids, e.g. decarboxylation of THCA and/or of CBDA, according to some embodiments. Some parameters relate to operation of the inhaler; and some relate to a structure of the chip, for example, an arrangement of the source material within the chip.

Chip and/or Source Material Related Parameters:
A density of the particles (1201) in the source material. In some embodiments, the particle density affects the passing of airflow in between particles.
A size of the particles (1203) in the source material. In some embodiments, pre-processing of the source material is performed to obtain a selected particle size or range. In an example, a cannabis grinding procedure is performed, using a cryogenic grinder. In an example, for cannabis, a particle size distribution obtained by a sieve analysis may include: D90: 550±150 μm, D50: 275±75 μm, D10: 120±60 μm, where D10, D50 and D90 are the intercepts for 10%, 50% and 90% of the cumulative mass, respectively. It is noted that the described sizes are provided only as an example, which may be especially suitable for Bedrocan grinding. Various cannabis strains may be processed to a variety of different sizes. Source materials in general may be naturally formed with and/or processed to other sizes than the ones described.
A molecular structure of substances (1205) in the source material. In some embodiments, the molecular structure may affect the chemical modification of the molecule, for example in response to heating. In some embodiments, the molecular structure may affect a boiling or vaporization temperature of the substance.
In some embodiments, molecules of a substance are synthesized with molecular groups of choice which may affect the chemical modification and/or vaporization temperature of the substance. For example, THCV is a cannabinoid that due to its molecular structure has a higher boiling point than, for example, Δ9-THC, and therefore additional thermal energy would need to be invested in order to vaporize the THCV. In this case, molecular groups of choice may be synthesized with THCV in order to lower its boiling point.
One or more substances added to the source material (1207), for example, substances added to preserve the material for longer; substances added to modify a vaporization temperature of one or more substances in the source material; substances added to affect (for example, accelerate or delay) decarboxylation directly; substances added to solidify the source material; substances added to liquefy the source material; substances added to extend shelf-life (e.g. beta-carotene, vitamin C, vitamin E), substances added to enhance vaporization (e.g. by increasing heat conductivity), such as vegetable glycerin, propanol glycol, MCTs.
A humidity level (1209) of the source material within the chip. In some embodiments, in high humidity conditions, vaporizing an active substance may require increasing the heating energy applied to the substance particles.
A type of carrier material (1211) within the chip, which may affect heating of the source material. In some embodiments, matrices comprising the source material are selected to include materials which may affect the release of active substances from the source material. Alternatively, matrices do not affect release and are solely used as a material in which the source material is absorbed or mounted. Such matrices may include cellulose and/or fibrous matrices.
The integrity of cellular and/or subcellular structures that are associated with and/or contain active substances. For example, in some embodiments extra energy may be required to cause vaporization of an active substance from an intact structure than for the same source material whilst the structure is not intact.
Permeability of the source material to airflow, which may affect the interplay between conduction and convection of heating energy within the source material.

Functional and Operation Related Parameters:
Heating-time profile (1213), for example as described herein.
Airflow-time profile (1215), for example as described herein.

Selection of Parameters

In some embodiments, one or more parameters are selected to compensate for one or more other parameters. Optionally, a trade-off between parameters is set to obtain decarboxylation efficiency within a certain range.

In some embodiments, by selecting a parameter while taking into account at least one other parameter, various constraints (e.g. system constraints, functional constraints, usage constraints) may be mitigated. Examples of considerations for parameter selection may include:
Particle size and density, airflow and heat: in some embodiments, a particle size may affect transferring of heat by convection and/or conduction within the source material, as well the removal or released active substances from the source material and as the ease of release of active substances from within the particles. Optionally, larger particles would allow for an increased flow of air between particles, for example as opposed to small particles which may be arranged in a denser manner. In some embodiments, the airflow rate is increased in order to compensate for the denser arrangement. In some embodiments, airflow is reduced in order to prevent overheating of particle surfaces due to convection.
airflow and user inhalation profile: in some embodiments, the rate of airflow is selected, on the one hand, to be sufficient for releasing and carrying the active substances away from the source material; and on the other hand, to be within a range suitable for inhalation by a user (for example, not too low, so as to allow the user to sense the flow; and not too high, which may require excessive effort by the user). In an example, the rate of flow is selected and/or controlled to be within the range of 0.5 L/min to 1.5 L/min, such as 0.75 L/min, 1 L/min, 1.3 L/min or intermediate, higher or lower rates.
In some embodiments, the airflow rate is set in response the user inhalation, optionally being modulated by use of the one or more bypass routes.
Heating duration and heating temperature: in some embodiments, lowering the temperature may be compensated for by increasing a duration of heating, and vice versa. Optionally, lowering the temperature allows for reducing the decarboxylation rate. Optionally, lowering the temperature allows for preserving a higher proportion of certain existing substances within the source material, such as terpenes.

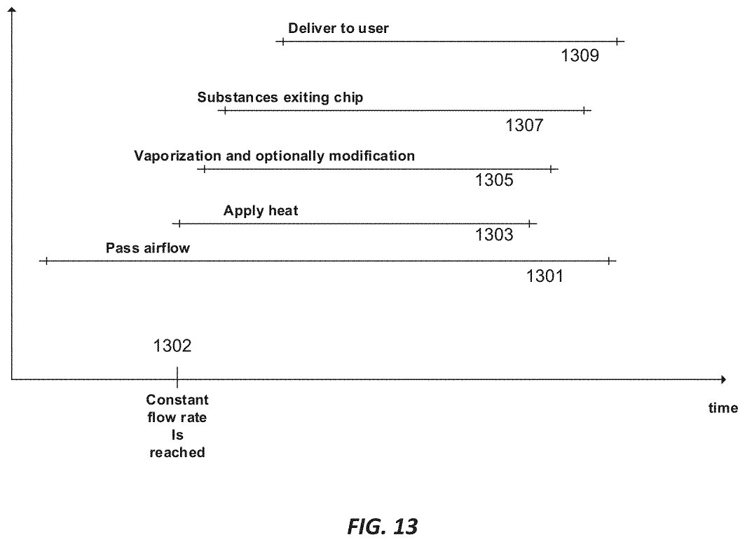

FIG. 13 is an example of a timeline for delivering using a selected operation profile one or more substances to a user, according to some embodiments.

It is noted that the timeline described by this figure is only a schematic example of a controlled process for release of one or more active substances from a source material, and should not be construed as limiting. The order of steps, a duration of each step, an overlap between steps, an indication for initiating and/or ceasing a process may vary for different operation profiles.

In some embodiments, air flow is allowed and/or directed to pass through the source material (1301). Optionally, airflow enters the inhaler in response to a user applying suction when placing their mouth on the inhaler and beginning to breathe in. In some embodiments, airflow passes through the source material and excess airflow passes through a bypass route, which does not pass through the source material.

In some embodiments, upon reaching a substantially, optionally pre-defined, constant rate of flow (1302) through the source material (as sensed, for example, by a flowrate or pressure sensor), heat is applied to the source material (1303). Almost immediately following the initiation of heat, one or more active substances may begin to be vaporized (1305) from the source material. Optionally, one or more of the active substances go through a modification occurring as result of heating and/or of passing airflow, for example, a chemical modification such as decarboxylation. Various heating and temperature profiles are for example as described in FIGS. 8 and 9 above.

In some embodiments, one or more substances are released only during the passing of airflow through the source material.

In some embodiments, an order in which two or more substances are released is controlled. In an example, a low temperature is applied (e.g. between 50-80° C.) to release terpenes; and then a higher temperature is applied, potentially releasing CBD and THC (or Δ9-THC) thereafter. In some embodiments, the released substances exit the chip (1307), for example by passing through the mesh and into one or more conduits extending to the inhaler mouthpiece, and the formed vapor/aerosol, including the one or more released active substances, is delivered to the user (1309).

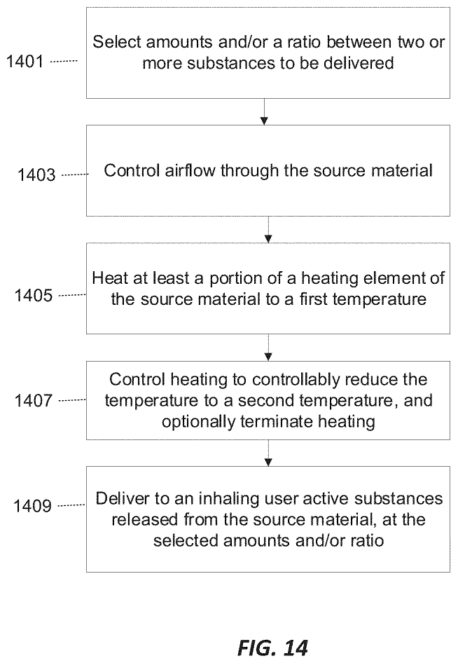

FIG. 14 is a flowchart of an operation profile in which heating is controlled according to a temperature gradient, according to some embodiments.

In some embodiments, an operation profile in which heating is controlled according to a temperature gradient is set for reaching a selected ratio and/or selected amounts of substances released (1401).

In some embodiments, the ratio is between a neutral cannabinoid and its precursor, for example, Δ9-THC and THCA. In some embodiments the ratio is between two cannabinoids or between a cannabinoid and a terpene. In some embodiments, a decarboxylation efficiency is selected). Decarboxylation efficiency is referred to herein, in accordance with some embodiments, as the percentage of: THCA divided by the total of (THCA+Δ9-THC).

In the operation profile described, in accordance with some embodiments, airflow through the source material is controlled (1403), optionally as described herein.

In some embodiments, heating is controlled as follows: at first, at least portion of a heating element, which heats the source material. is heated to a first temperature (1405). In an example, the at least a portion comprises first and/or second surfaces of a mesh which extends across the source material.

Then, in some embodiments, heating is controlled to reduce the temperature to a second temperature (1407), optionally terminating heating upon reaching the second temperature. Alternatively heating may be maintained or even increased rather than terminating. In some embodiments control of heating is carried out by the inhaler controller, using an indication from a temperature sensor configured in or on the source material and/or in or on the heating element, for example on one or both of the mesh surfaces.

In some embodiments, heating is controlled so that the temperature gradually decreases.

In some embodiments, heating is controlled to maintain a temperature of the source material within a selected range, for example, within a range selected according to the vaporization temperature of one or more active substances (for example, within 50° C., 70° C., 30° C. or intermediate, higher or lower temperatures). In some embodiments, heating is controlled to maintain a temperature of the source material within a range in which the selected decarboxylation efficiency is reached. Optionally, heating and/or airflow are controlled to lengthen or instead shorten a time period over which decarboxylation is likely to occur.

In some embodiments, the active substances released from the source material are delivered to the inhaling user at the selected ratio and/or amounts (1409). In an example, THCA and Δ9-THC are delivered to the user at a selected ratio or range thereof; CBD and CBDA are delivered to the user at a selected ratio or range thereof; different terpenes are delivered to the user at a selected ratio or range thereof.

In the following experiment performed by the inventors, 46 samples of 13.5 mg Bedrocan Cannabis were tested using the operation profile described in FIG. 14. In this experiment, heating was applied for a total 1650 ms. The first temperature was set to 195° C., and the second temperature was set for 175° C. As can be observed, a substantially constant decarboxylation efficiency was obtained, ranging between 9%-13% (calculated as the percentage of THCA out of the sum of THCA and Δ9-THC.)

A potential advantage of heating to obtain a temperature gradient may include bringing the source material controllably to a state in which decarboxylation occurs substantially at the pre-selected efficiency and maintaining it in that state. In some embodiments, for different temperature gradients, different decarboxylation efficiencies can be reached.

TABLE 4

| | | % THCA/ | TOTAL | |
|---|---|---|---|---|
| | | TOTAL | (THCA + | |
| sample | THCA | (Δ9-THC + | Δ9-THC) | Δ9-THC |
| # | [mg] | THCA) | [mg] | [mg] |
| 1 | 0.065 | 11% | 0.585 | 0.514 |
| 2 | 0.071 | 13% | 0.562 | 0.485 |
| 3 | 0.069 | 13% | 0.548 | 0.472 |
| 4 | 0.074 | 13% | 0.572 | 0.491 |
| 5 | 0.068 | 13% | 0.540 | 0.466 |
| 7 | 0.070 | 11% | 0.613 | 0.536 |
| 8 | 0.075 | 11% | 0.659 | 0.577 |
| 11 | 0.067 | 11% | 0.604 | 0.531 |
| 12 | 0.076 | 12% | 0.634 | 0.550 |
| 13 | 0.074 | 12% | 0.609 | 0.527 |
| 14 | 0.070 | 10% | 0.688 | 0.611 |
| 15 | 0.082 | 12% | 0.684 | 0.594 |
| 16 | 0.070 | 11% | 0.621 | 0.545 |
| 17 | 0.074 | 12% | 0.628 | 0.547 |
| 18 | 0.077 | 12% | 0.646 | 0.562 |
| 20 | 0.076 | 11% | 0.698 | 0.615 |
| 21 | 0.082 | 12% | 0.679 | 0.588 |
| 22 | 0.085 | 12% | 0.707 | 0.613 |
| 23 | 0.079 | 12% | 0.666 | 0.580 |
| 24 | 0.082 | 12% | 0.706 | 0.615 |
| 26 | 0.082 | 13% | 0.649 | 0.558 |
| 27 | 0.077 | 13% | 0.609 | 0.524 |
| 28 | 0.081 | 13% | 0.609 | 0.521 |
| 29 | 0.072 | 11% | 0.648 | 0.569 |
| 30 | 0.078 | 13% | 0.585 | 0.500 |
| 32 | 0.064 | 10% | 0.607 | 0.537 |
| 33 | 0.076 | 11% | 0.711 | 0.628 |
| 34 | 0.083 | 12% | 0.714 | 0.623 |
| 35 | 0.076 | 11% | 0.699 | 0.616 |
| 36 | 0.081 | 11% | 0.733 | 0.644 |
| 37 | 0.081 | 11% | 0.712 | 0.624 |
| 38 | 0.072 | 11% | 0.630 | 0.551 |
| 39 | 0.083 | 12% | 0.697 | 0.605 |
| 40 | 0.079 | 11% | 0.698 | 0.612 |
| 41 | 0.081 | 12% | 0.663 | 0.574 |
| 42 | 0.069 | 9% | 0.779 | 0.703 |
| 43 | 0.084 | 11% | 0.731 | 0.639 |
| 44 | 0.084 | 11% | 0.755 | 0.662 |
| 45 | 0.075 | 12% | 0.637 | 0.554 |
| 46 | 0.083 | 11% | 0.729 | 0.638 |

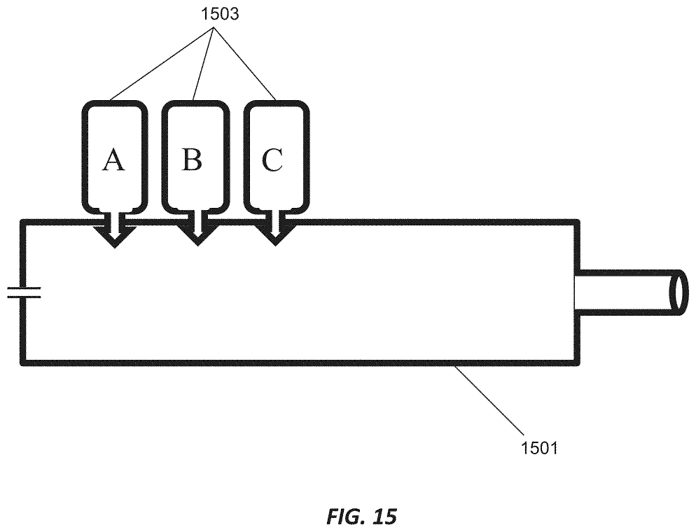

FIG. 15 is a schematic diagram of an inhaler configured to deliver, optionally in a single inhalation of the user, two or more active substances at predefined amounts and/or at a known ratio, according to some embodiments.

In some embodiments, an inhaler 1501 comprises a plurality of independently accessible reservoirs 1503 (e.g. 2, 3, 4, 5, 6, 10 reservoirs), each reservoir comprising a source material (marked as A, B, C) from each of which one or more active substances can be released.

In some embodiments, the one or more active substances are vaporized. Vaporization of the active substances from the reservoirs may be carried out via different methods, which involve heating the source material and generating a vapor, aerosol or spray, which is delivered to the user via inhalation. Such methods may include, for example: heating the source material by heating a surrounding structure (e.g. heating at least a portion of a housing containing the source material, heating a surface which the source material are in contact with, heating a substrate on which the source material are found); heating a structure on which a film or coating of or source material is mounted; melting the source material; aerosolizing a liquid source material.

In some embodiments, the one or more active substances are released from the respective reservoirs 1503 by any means known in the art, including by dispensing a powder and/or actuating a pressure canister. The amount of each released substance may be controlled for example by controlling a number of small doses actuated from each reservoir 1305.

In some embodiments, the reservoirs 1503 are independently accessible. Optionally, the source material of each reservoir is configured to be used without affecting the other reservoirs.

In some embodiments, in use, one or more active substances are released from the reservoirs, optionally simultaneously. In some embodiments, the inhaler is actuated to deliver the active substances at predefined selected amounts and/or at a predefined ratio.

In some embodiments, the different amounts and/or ratio are generated by selecting a specific operation profile, including for example a heating profile and/or an airflow profile, such as described hereinabove. In some embodiments, at least one of the active substances is delivered at a pre-defined, optionally constant amount, while one or more other substances are not constant, and optionally vary in response to selection of a different operation profile.

It is noted that the source material of the reservoir may take the form of a solid, liquid, paste, powder, granulated particles, natural plant form, extract, isolate. In some embodiments, different reservoirs include a source material in different forms, for example, a first reservoir may include a liquid extract, and a second reservoir may include granulated particles. In some embodiments, active substances from reservoirs including materials in different forms are released simultaneously, for example to be delivered to the user during a single inhalation.

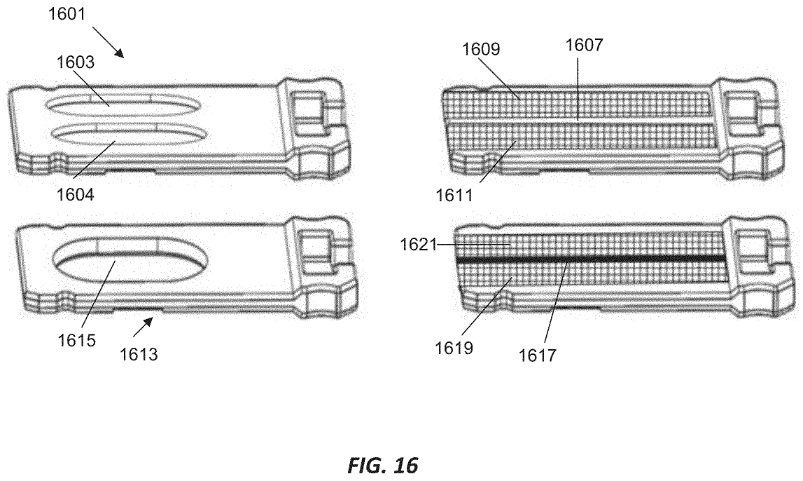

FIG. 16 schematically illustrates source material chips comprising individually controllable portions, according to some embodiments.

In some embodiments, the inhaler is configured to independently control distinct portions of the source material. Optionally, a different operation profile is applied to different portions.

In the first example illustrated, a source material chip 1601 comprises two cavities 1603, 1604 each cavity for accepting a source material. As shown for chip 1607, each cavity may be in association with heating element 1609, 1611, respectively, essentially as shown in FIGS. 1C-1D for the cavity 165 and heating element 114. Optionally each of heating elements 1609, 1611 is U-shaped and spans the respective cavity 1603, 1604 on both sides. Optionally, the heating element is in the form of a mesh, extending across the layer of source material, optionally on both sides of the layer.

In the second example illustrated, a source material chip 1613 is essentially like chip 160 of FIGS. 1C-1D comprises a single cavity 1615 and has a single heating element 1617 covering the cavity 1615 (possibly a single U-shaped heating element, covering both sides). However, in the example shown herein (FIG. 16) heating element includes a separation 1617 defined therein, dividing the heating element into two separate portions 1619, 1621.

During use, in some embodiments, airflow and/or heating are controlled to affect only one of the heating elements (e.g. one of 1609, 1611; one of 1619, 1621), and the respective portions. In some embodiments, heating is controlled such that each portion is heated according to a different heating profile (e.g. a different temperature, a different duration). Optionally, each portion is configured to receive electrical current independently of other portions, so as to be heated separately. Optionally, only one heating element is heated. In some embodiments, the electrical current is conducted to the heating element via an electrode or wire.

In some embodiments, airflow is controlled such that airflow is passed through each of the portions at a different rate and/or throughout a different duration.

In some embodiments, the source material is defined as a plurality of portions, such as 2, 3, 4, 5, 6, 10, or intermediate, larger or smaller number of portions. In some embodiments, different portions include different source materials or a mixture of source materials. In an example, different portions include cannabis having different active substance concentrations, such as THC, CBD contents. In another example, a portion includes cannabis, and another portion includes tobacco. Alternatively, different portions include similar source material. Optionally one or more portions includes one or more of botanical substances selected from the group consisting of Cannabis sativa, Cannabis indica, Cannabis ruderalis, Acacia spp., Amanita muscaria, Yage, Atropa belladonna, Areca catechu, Brugmansia spp., Brunfelsia latifolia, Desmanthus illinoensis, Banisteriopsis caapi, Trichocereus spp., Theobroma cacao, Capsicum spp., Cestrum spp., Erythroxylum coca, Solenostemon scutellarioides, Arundo donax, Coffea arabica, Datura spp., Desfontainia spp., Diplopterys cabrerana, Ephedra sinica, Claviceps purpurea, Paullinia cupana, Argyreia nervosa, Hyoscyamus niger, Tabernanthe iboga, Lagochilus inebriens, Justicia pectoralis, Sceletium tortuosum, Piper methysticum, Catha edulis, Mitragyna speciosa, Leonotis leonurus, Nymphaea spp., Nelumbo spp., Sophora secundiflora, Mucuna pruriens, Mandragora officinarum, Mimosa tenuiflora, Ipomoea violacea, Psilocybe spp., Panaeolus spp., Myristica fragrans, Turbina corymbosa, Passiflora incarnata, Lophophora williamsii, Phalaris spp., Duboisia hopwoodii, Papaver somniferum, Psychotria viridis, spp., Salvia divinorum, Combretum quadrangulare, Trichocereus pachanoi, Heimia salicifolia, Stipa robusta, Solandra spp., Hypericum perforatum, Peganum harmala, Tabernaemontana spp., Camellia sinensis, Nicotiana tabacum, Nicotiana rustica, Virola theidora, Voacanga africana, Lactuca virosa, Artemisia absinthium, Ilex paraguariensis, Anadenanthera spp., Corynanthe yohimbe, Calea zacatechichi, Coffea spp. (Rubiaceae), Sapindaceae spp., Camellia spp., Malvaceae spp., Aquifoliaceae spp., Hoodia spp. Chamomilla recutita, Passiflora incarnate, Camellia sinensis, Mentha piperita, Mentha spicata, Rubus idaeus, Eucalyptus globulus, Lavandula officinalis, Thymus vulgaris, Melissa officinalis, Tobacco, Aloe Vera, Angelica, Anise, Ayahuasca (Banisteriopsis caapi), Barberry, Black Horehound, Blue Lotus, Burdock, Camomille/Chamomile, Caraway, Cat's Claw, Clove, Comfrey, Corn Silk, Couch Grass, Damiana, Damiana, Dandelion, Ephedra, Eucalyptus, Evening Primrose, Fennel, Feverfew, Fringe Tree, Garlic, Ginger, Ginkgo, Ginseng, Goldenrod, Goldenseal, Gotu Kola, Green Tea, Guarana, Hawthorn, Hops, Horsetail, Hyssop, Kola Nut, Kratom, Lavender, Lemon Balm, Licorice, Lion's Tail (Wild Dagga), Maca Root, Marshmallow, Meadowsweet, Milk Thistle, Motherwort, Passion Flower, Passionflower, Peppermint, Prickly Poppy, Purslane, Raspberry Leaf, Red Poppy, Sage, Saw Palmetto, Sida Cordifolia, Sinicuichi (Mayan Sun Opener), Spearmint, Sweet Flag, Syrian Rue (Peganum harmala), Thyme, Turmeric, Valerian, Wild Yam, Wormwood, Yarrow, Yerba Mate, and Yohimbe.

In the example described by Table 5 below, 3 different operation profiles are configured to deliver from separate portions the same total amount of an active substance “A”, with a different composition of other substances released (“a”, “B”), by controlling the ratio released from at least one of the separate portions (such as the “A”:“a” ratio, “A”:“B” ratio).

In some embodiments, by implementing a different operation profile, the total aerosol composition is modified. Optionally, as shown in the below table, a total amount of at least one of the active substances is optionally maintained constant.

In a specific example, substance “A” is Δ9-THC, substance “a” is THCA, substance “B” is CBD.

TABLE 5

| Source Material | | | | |
|---|---|---|---|---|
| (Active | A | A | B | Aerosol |
| Substances) | (A & a) | (A & a) | (A & B) | (total) |
| Operation | 1A:1a | 1A:1a | 1A:0B | 3A:2a:0B |
| Profile 1 | | | | |
| Operation | 1A:1a | 1A:2a | 1A:0B | 3A:3a:0B |
| Profile 2 | | | | |
| Operation | 1A:2a | 1A:1a | 1A:3B | 3A:3a:3B |
| Profile 3 | | | | |

Systems, devices and/or methods for example as described in PCT publications WO2016001925, WO2016001926, WO2016001921, WO2017122196, WO2017122201 incorporated herein by reference, are also contemplated by the present application. Methods for analyzing and/or using information (such as information collected from one user or from a plurality of users) for example as described in PCT publications WO2016001925, WO2016001926, WO2016001921, WO2017122196, WO2017122201 are also contemplated by the present application.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method for providing, during a single inhalation from an inhaler, a controllable ratio between at least a first active substance and a second active substance provided from the same distinct source material portion, comprising:
defining a ratio between the first and second active substances to be delivered to a user;
setting operation parameters in accordance with the ratio, the operation parameters including at least one of a heating profile of the source material portion and an airflow profile through the source material portion; and
operating the inhaler according to the operation parameters to deliver to the user the at least first and second substances together during a single inhalation, the at least first and second active substances released from the same distinct source material portion at the defined ratio.

2. The method according to claim 1, wherein the defining includes defining an amount of at least the first substance to be delivered to the user during the single inhalation.

3. The method according to claim 1, wherein the defining includes defining a ratio between the first and second active substances.

4. The method according to claim 1, wherein a change in operation parameters changes an efficiency of at least one of: vaporization of at least one of the first active substance and the second active substance, and a chemical modification of at least one of the first active substance and the second active substance.

5. The method according to claim 1, wherein the second substance is a chemical derivative of the first substance.

6. The method according to claim 1, wherein the source material includes at least one of: a botanical material (e.g. plant and/or fungus), two or more different botanical materials, a synthetic material, a synthetic material and a botanical material.

7. The method according to claim 1, wherein the first substance is at least one of: Δ9-tetrahydrocannabinol (Δ9-THC), tetrahydrocannabinol acid (THCA) and Cannabidiol (CBD) and the second substance is at least one of a cannabinoid different than the first substance and a terpene.

8-12. (canceled)

13. The method according to claim 1, wherein the heating profile is set to release the first active substance and the second active substance simultaneously into aerosol formed by the inhaler.

14. An inhaler for delivering at least two active substances released from a same distinct source material portion to an inhaling user, the inhaler comprising:
a controller pre-programmed with and/or in communication with a storage including a plurality of operation profiles; and
at least one of:
at least one conductor configured to supply sufficient energy for heating the source material portion when the source material portion is received within the inhaler; and
an airflow system for passing airflow through the source material portion and for delivering together the at least two active substances released from the same distinct source material portion to an inhaling user, in a single inhalation;
wherein each of the plurality of operation profiles is associated with a different ratio between the at least two active substances.

15. The inhaler according to claim 14, wherein each of the operation profiles is associated with a different therapeutic and/or psychoactive and/or adverse effect the ratio is expected to have on the user.

16. The inhaler according to claim 15, wherein the different effect includes a different magnitude of a similar effect.

17. The inhaler according to claim 14, comprising a user interface configured to receive feedback input from the user and to automatically select an operation profile in response to the feedback input.

18. The inhaler according to claim 14, wherein the controller is configured for setting two or more operation profiles for use during a single inhalation, the two or more operation profiles selected according to amounts of the active substances or a ratio between the active substances to be released from the source material portion and delivered to the user.

19. The inhaler according to claim 18, wherein the two or more operation profiles are configured to deliver the same amount of a first active substance to the user.

20. The inhaler according to claim 14, wherein the storage is configured on a cellular phone application.

21. The inhaler according to claim 14, wherein the storage is cloud based.

22. The inhaler according to claim 14, wherein each of the plurality of operation profiles is configured to operate within a single inhalation.

23. A method for delivering to a user, via an inhaler, a known ratio between at least one cannabinoid and the acidic form of that cannabinoid, the method comprising:
defining a ratio to be delivered of the at least one cannabinoid and the acidic form of that cannabinoid;
setting operation parameters in accordance with the defined ratio, the operation parameters including at least one of a heating-time profile and an airflow-time profile to be applied to a source material containing at least the acidic form of the cannabinoid;
operating the inhaler according to the operation parameters to deliver to the user the at least one cannabinoid and the acidic form of that cannabinoid at the defined ratio.

24. The method according to claim 23, wherein the at least one cannabinoid is Δ9-THC and the acidic form of that cannabinoid is THCA; or wherein the at least one cannabinoid is CBD and the acidic form of that cannabinoid is CBDA.

25. (canceled)

26. The method according to claim 23, wherein the ratio is defined individually per the user, according to one or both of a treatment plan, a desired therapeutic effect.

27-28. (canceled)

29. The method according to claim 23, comprising setting operation parameters which increase vaporization of the acidic form before decarboxylation.

30. The method according to claim 23, wherein increasing the rate of airflow through the source material, while maintaining other parameters constant, increases the ratio between the acidic form and the total of: the at least one cannabinoid and the acidic form of that cannabinoid.

31. The method according to claim 23, wherein raising the heating temperature, while maintaining other parameters constant, reduces the ratio between the acidic form and the total of: the at least one cannabinoid and the acidic form of that cannabinoid.

32. The method according to claim 23, wherein lengthening the heating duration, while maintaining other parameters constant, reduces the ratio between the acidic form and the total of: the at least one cannabinoid and the acidic form of that cannabinoid.

33-43. (canceled)
